(19) United States
(12) Patent Application Publication
(10) Pub. No.: US 2014/0204196 A1
(43) Pub. Date: Jul. 24, 2014

(54) FOCUS AND IMAGING SYSTEM AND TECHNIQUES USING ERROR SIGNAL

(71) Applicants: Gregory C. Loney, Los Altos, CA (US); Glenn Stark, Scott Valley, CA (US)

(72) Inventors: Gregory C. Loney, Los Altos, CA (US); Glenn Stark, Scott Valley, CA (US)

(73) Assignee: VENTANA MEDICAL SYSTEMS, INC., Tucson, AZ (US)

(21) Appl. No.: 14/237,050

(22) PCT Filed: Aug. 21, 2012
(86) PCT No.: PCT/EP2012/066265

§ 371 Date: Feb. 4, 2014

Related U.S. Application Data
(60) Provisional application No. 61/532,709, filed on Sep. 9, 2011.

(51) Int. Cl. A61B 1/00

(52) U.S. Cl. CPC A61B 1/00188
USPC 348/80

(57) ABSTRACT

Systems and techniques for an optical scanning microscope and/or other appropriate imaging system includes components for scanning and collecting focused images of a tissue sample and/or other object disposed on a slide. The focusing system described herein provides for determining best focus for each snapshot as a snapshot is captured, which may be referred to as “on-the-fly focusing.” Best focus may be determined using an error function generated according to movement of a dither focusing lens. The devices and techniques provided herein lead to significant reductions in the time required for forming a digital image of an area in a pathology slide and provide for the creation of high quality digital images of a specimen at high throughput.

### TECHNICAL FIELD

This application relates to the field of imaging and, more particularly, to systems and techniques for obtaining and capturing images.

### BACKGROUND OF THE INVENTION

Molecular imaging identification of changes in the cellular structures indicative of disease remains a key to the better understanding in medicinal science. Microscopy applications are applicable to microbiology (e.g., gram staining, etc.), plant tissue culture, animal cell culture (e.g. phase contrast microscopy, etc.), molecular biology, immunology (e.g., ELISA, etc.), cell biology (e.g., immunofluorescence, chromosome analysis, etc.), confocal microscopy, time-lapse and live cell imaging, series and three-dimensional imaging.

There have been advances in confocal microscopy that have unraveled many of the secrets occurring within the cell and the transcriptional and translational level changes can be detected using fluorescence markers. The advantage of the confocal approach results from the capability to image individual optical sections at high resolution in sequence through the specimen. However, there remains a need for systems and methods for digital processing of images of pathological tissue that provide accurate analysis of pathological tissues, at a relatively low cost.

It is a desirable goal in digital pathology to obtain high resolution digital images for viewing in a short period of time. Current manual methods whereby the pathologist views a slide through the ocular lens of a microscope allows a diagnosis upon inspection of cell characteristics or count of stained cells vs. unstained cells. Automated methods are desirable whereby digital images are collected, viewed on high resolution monitors and may be shared and archived for later use. It is advantageous that the digitization process be accomplished efficiently at a high throughput and with high resolution and high quality images.

In conventional virtual microscopy systems, imaging techniques can produce individual images that may be significantly out of focus over much of the image. Conventional imaging systems are restricted to a single focal distance for each individual snapshot taken by a camera, thus, each of these “fields of view” has areas that are out of focus when the subject specimen being scanned does not have a uniform surface. At the high magnification levels employed in virtual microscopy, specimens with a uniform surface are extremely rare.

Conventional systems use a pre-focusing technique to address the high proportion of out-of-focus images that is based on a two-step process that includes: 1) determining, in a first pass, the best focus at an array of points, separated by n image frames, arranged on a two-dimensional grid laid on the top of a tissue section; and 2) in another pass, moving to each focus point and acquire an image frame. For points between these best focus points, the focus is interpolated. While this two-step process may reduce or even eliminate out-of-focus images, the process results in a significant loss in the speed of acquiring the tiled images.

Accordingly, it would be desirable to provide a system that overcomes the significant problems inherent in conventional imaging systems and efficiently provides focused, high quality images at a high throughput.

### SUMMARY OF THE INVENTION

According to the system described herein, a device for obtaining a focused image of a specimen includes an objective lens disposed for examination of the specimen. A slow focusing stage may be coupled to the objective lens, in which the slow focusing stage controls movement of the objective lens. A dither focus stage including a dither lens and the dither focus stage may move the dither lens. A focus sensor may provide focus information in accordance with light transmitted via the dither lens. At least one electrical component may use the focus information to determine a metric and a first focus position of the objective lens in accordance with the metric. The at least one electrical component may include an error signal component that processes error signal information generated based on the metric to determine the first focus position. The at least one electrical component may send position information to the slow focusing stage for moving the objective lens into the first focus position. An image sensor may capture an image of the specimen after the objective lens is moved into the first focus position. The error signal information may be determined according to an error signal function using points of a waveform generated based on the metric according to the motion of the dither lens. The error signal function may be a contrast error signal function, and the first focus position may be determined where the contrast error signal function is zero. The contrast error signal function may be determined based on at least three points of a sharpness waveform computed for each of at least one position on a sharpness response curve where the motion of the dither lens is centered. The contrast error signal (CES) may be represented by an equation: CES=(a−c)/b, where a is a trough of the sharpness waveform, b is a peak of the sharpness waveform, and c is a subsequent trough of the sharpness waveform. An XY moving stage may be provided, on which the specimen is disposed, and the at least one electrical component may control movement of the XY moving stage and/or the XY moving stage may be phase locked with the motion of the dither lens. The dither focus stage may include a voice-coil actuated flexured assembly that moves the dither lens in a translational motion. The dither lens may be moved at a resonant frequency that is at least 60 Hz, and wherein the at least one electrical component uses the focus information to perform at least 60 focus calculations per second. The focus sensor and the dither focus stage may be set to operate bidirectionally and the focus sensor may produce the focus information on both an up and down portion of a sinusoid waveform of the motion of the dither lens at the resonant frequency. The metric may include at least one of: contrast information, sharpness information, and chroma information.

According further to the system described herein, a method for obtaining a focused image of a specimen is provided. The method may include controlling movement of an objective lens disposed for examination of the specimen. Motion of a dither lens may be controlled. Focus information may be provided in accordance with light transmitted via the dither lens. The focus information may be used to determine a metric and determine a first focus position of the objective lens in accordance with the metric. Determining the first focus position may include processing error signal information generated based on the metric. Position information may be sent that is used to move the objective lens into the first focus position. The error signal information may be determined according to an error signal function using points of a waveform generated based on the metric according to the motion of the dither lens. The error signal function may be a contrast error signal function, and the first focus position may be determined where the contrast error signal function is zero. The contrast error signal function may be determined based on at least three points of a sharpness waveform computed for each of at least one position on a sharpness response curve where the motion of the dither lens is centered. The contrast error signal (CES) may be represented by an equation: CES=(a−c)/b, where a is a trough of the sharpness waveform, b is a peak of the sharpness waveform, and c is a subsequent trough of the sharpness waveform. The first focus position may be determined as a best focus position, and the method may further include capturing an image of the specimen after the objective lens is moved into the best focus position. The dither lens may be moved at a resonant frequency that is at least 60 Hz, and at least 60 focus calculations may be performed per second. The metric may include at least one of: sharpness information, contrast information and chroma information.

According further to the system described herein, a non-transitory computer readable medium stores software for obtaining a focused image of a specimen. The software may include executable code that controls movement of an objective lens disposed for examination of the specimen. Executable code may be provided that controls motion of a dither lens. Executable code may be provided that provides focus information in accordance with light transmitted via the dither lens. Executable code may be provided that uses the focus information to determine a metric and determine a first focus position of the objective lens in accordance with the metric. Determining the first focus position may include processing error signal information generated based on the metric. Executable code may be provided that sends position information that is used to move the objective lens into the first focus position. The error signal information may be determined according to an error signal function using points of a waveform generated based on the metric according to the motion of the dither lens. The error signal function may be a contrast error signal function, and the first focus position may be determined where the contrast error signal function is zero. The contrast error signal function may be determined based on at least three points of a sharpness waveform computed for each of at least one position on a sharpness response curve where the motion of the dither lens is centered. The contrast error signal (CES) may be represented by an equation: CES=(a−c)/b, where a is a trough of the sharpness waveform, b is a peak of the sharpness waveform, and c is a subsequent trough of the sharpness waveform.

### BRIEF DESCRIPTION OF THE DRAWINGS

Embodiments of the system described herein will be explained in more detail herein based on the figures of the drawings, which are briefly described as follows.

FIG. 1 is a schematic illustration of an imaging system of a scanning microscope and/or other scanning device that may include various component devices used in connection with digital pathology sample scanning and imaging according to various embodiments of the system described herein.

FIG. 2 is a schematic illustration showing an imaging device including a focus system according to an embodiment of the system described herein.

FIGS. 3A and 3B are schematic illustrations of an embodiment of the control system showing that the control system may include appropriate electronics.

FIG. 4 is a schematic illustration showing the dither focus stage in more detail according to an embodiment of the system described herein.

FIGS. 5A-5E are schematic illustrations showing an iteration of the focusing operations according to the system described herein.

FIG. 6A is a schematic illustration of a plot showing the command waveform of the dither focus optics and sharpness determinations according to an embodiment of the system described herein.

FIG. 6B is a schematic illustration showing a plot of calculated sharpness (Zₛ) values for a portion of the sine wave motion of the dither lens.

FIGS. 7A and 7B are schematic illustrations showing focusing determinations and adjustments of a specimen (tissue) according to an embodiment of the system described herein.

FIG. 8 is a schematic illustration showing a camera window with image frame and focus frame in connection with focus processing and imaging according to an embodiment of the system described herein.

FIG. 9 is a schematic illustration showing an example of a sharpness profile including a sharpness curve and contrast error signal for each sharpness response at multiple points that are sampled by the dither focusing optics according to an embodiment of the system described herein.

FIG. 10 shows a functional control loop block diagram illustrating use of the contrast function to produce a control signal to control the slow focus stage.

FIG. 11 is a schematic illustration showing the focus frame being broken up into zones in connection with focus processing and imaging according to an embodiment of the system described herein.

FIGS. 12A and 12B show graphical illustrations of different sharpness values that may be obtained at points in time for embodiments in accordance with techniques herein.

FIG. 13 is a flow diagram showing on-the-fly focus processing during scanning of a specimen under examination according to an embodiment of the system described herein.

FIG. 14 is flow diagram showing processing at the slow focus stage according to an embodiment of the system described herein.

FIG. 15 is a flow diagram showing image capture processing according to an embodiment of the system described herein.

FIG. 16 is a schematic illustration showing an alternative arrangement for focus processing according to an embodiment of the system described herein.

FIG. 17 is a schematic illustration showing an alternative arrangement for focus processing according to another embodiment of the system described herein.

FIG. 18 is a flow diagram showing processing to acquire a mosaic image of tissue on a slide according to an embodiment of the system described herein.

FIG. 19 is a schematic illustration showing an implementation of an precision stage (e.g., a Y stage portion) of an XY stage that may be used in connection with an embodiment of the system described herein.

FIGS. 20A and 20B are more detailed views of the moving stage block of the precision stage that may be used in connection with an embodiment of the system described herein

FIG. 21 shows an implementation of an entire XY compound stage according to the precision stage features discussed herein and including a Y stage, an X stage and a base plate that may be used in connection with an embodiment of the system described herein.

FIG. 22 is a schematic illustration showing an illumination system for illuminating a slide using a light-emitting diode (LED) illumination assembly that may be used in connection with an embodiment of the system described herein.

FIG. 23 is a schematic illustration showing a more detailed view of an embodiment for a LED illumination assembly that may be used in connection with an system described herein.

FIG. 24 is a schematic showing an exploded view of a specific implementation of an LED illumination assembly that may be used in connection with an embodiment of the system described herein.

### DETAILED DESCRIPTION OF VARIOUS EMBODIMENTS

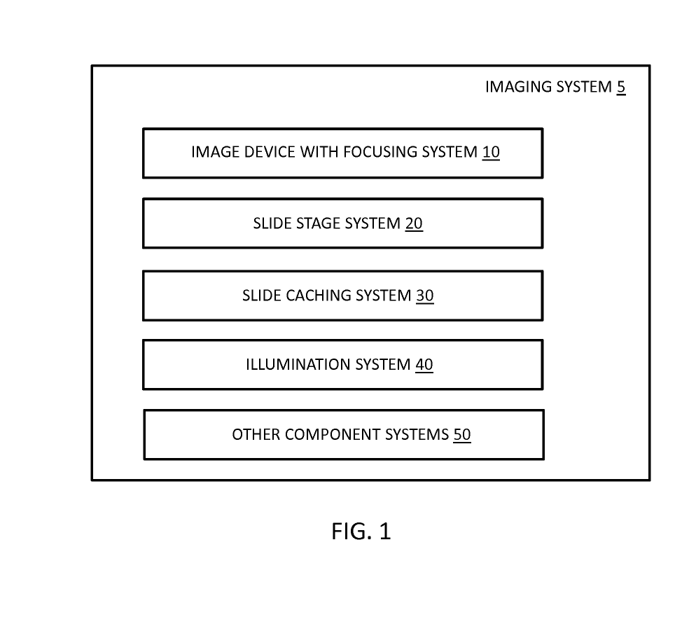

FIG. 1 is a schematic illustration of an imaging system 5 of a scanning microscope and/or other scanning device that may include various component devices used in connection with digital pathology sample scanning and imaging according to various embodiments of the system described herein. The imaging system 5 may include an imaging device with a focusing system 10 according to embodiments further discussed elsewhere herein. Additionally, in various embodiments, the imaging system 5 may include other systems used in connection imaging or other appropriate operations, including one or more of a slide stage system 20, a slide caching system 30 and an illumination system 40, among other component systems 50, as further discussed in detail elsewhere herein. Reference is made to WO 2011/049608 to Loney et al. entitled “Imaging System and Techniques,” which is incorporated herein by reference, that describes examples of various component systems and techniques that may be used for imaging and other appropriate operations, particularly for microscopy imaging. It is also noted that the system described herein may be used in connection with microscope slide scanning instrument architectures and techniques for image capture, stitching and magnification as described in U.S. Patent App. Pub. No. 2008/0240613 A1 to Dietz et al., entitled “Digital Microscope Slide Scanning System and Methods,” which is incorporated herein by reference, including features in connection with reconstituting an image with a magnification without substantial loss of accuracy and displaying or storing the reconstituted image.

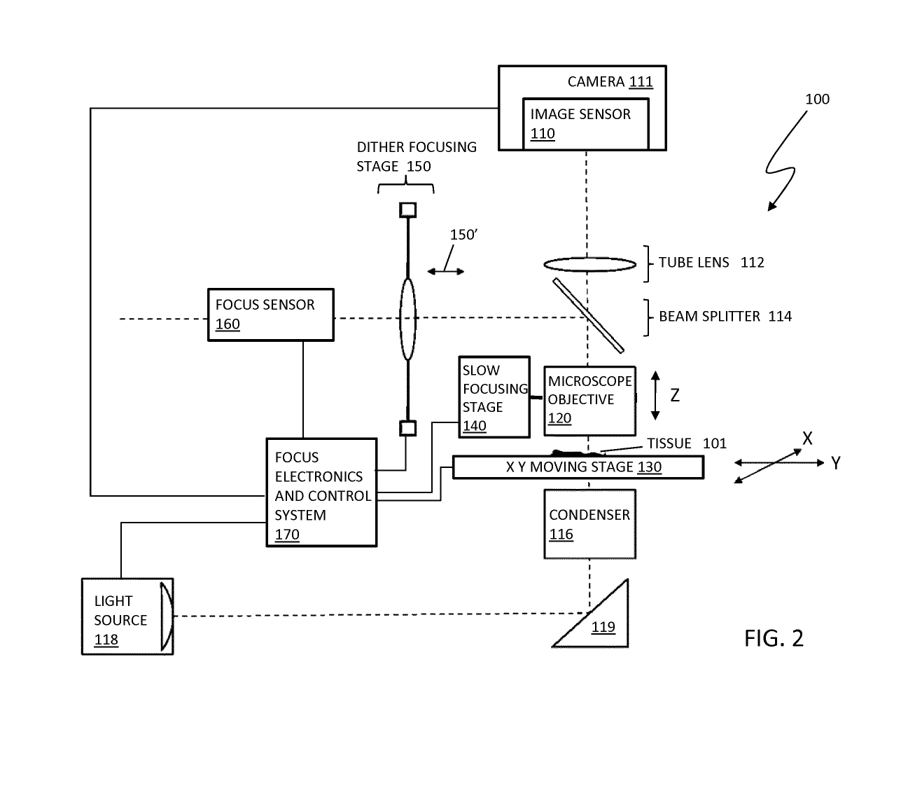

FIG. 2 is a schematic illustration showing an imaging device 100 of an optical scanning microscope and/or other appropriate imaging system that includes components of a focusing system for taking focused images of a tissue sample 101 and/or other object disposed on a slide according to an embodiment of the system described herein. The focusing system described herein provides for determining best focus for each snapshot as a snapshot is captured, which may be referred to as “on-the-fly focusing.” The devices and techniques provided herein lead to significant reductions in the time required for forming a digital image of an area in a pathology slide. The system described herein integrates steps of the two-step approach of conventional systems and essentially eliminates the time required for pre-focusing. The system described herein provides creating a digital image of a specimen on a microscope slide using on-the-fly processing for capturing snapshots in which the total time for capturing all the snapshots is less than the time required by a method using a step of predetermining focus points for each snapshot prior to capturing the snapshots.

The imaging device 100 may include an imaging sensor 110, such as a charge-coupled device (CCD) and/or complimentary metal-oxide semiconductor (CMOS) image sensor, that may be part of a camera 111 that captures digital pathology images. The imaging sensor 110 may receive transmitted light from a microscope objective 120 transmitted via a tube lens 112, a beam splitter 114 and including other components of a transmitted light microscope such as a condenser 116 and a light source 118 and/or other appropriate optical components 119. The microscope objective 120 may be infinity-corrected. In one embodiment, the beam splitter 114 may provide for apportioning approximately 70% of the light beam source directed to the image sensor 110 and the remaining portion of approximately 30% directed along a path to the dither focusing stage 150 and focus sensor 160. The tissue sample 101 being imaged may be disposed on an XY moving stage 130 that may be moved in X and Y directions and which may be controlled as further discussed elsewhere herein. A slow focusing stage 140 may control movement of the microscope objective 120 in the Z direction to focus an image of the tissue 101 that is captured by the image sensor 110. The slow focusing stage 140 may include a motor and/or other suitable device for moving the microscope objective 120. A dither focusing stage 150 and a focus sensor 160 are used to provide fine focusing control for the on-fly-focusing according to the system described herein. In various embodiments, the focus sensor 160 may be a CCD and/or CMOS sensor.

The dither focusing stage 150 and the focus sensor 160 provide on-the-fly focusing according to sharpness values and/or other metrics that are rapidly calculated during the imaging process to obtain a best focus for each image snapshot as it is captured. As further discussed in detail elsewhere herein, the dither focusing stage 150 may be moved at a frequency, e.g., in a sinusoidal motion, that is independent of and exceeds the movement frequency practicable for the slower motion of the microscope objective 120. Multiple measurements are taken by the focus sensor 160 of focus information for views of the tissue over the range of motion of the dither focusing stage 150. The focus electronics and control system 170 may include electronics for controlling the focus sensor and dithering focus stage 150, a master clock, electronics for controlling the slow focus stage 140 (Z direction), X-Y moving stage 130, and other components of an embodiment of a system in accordance with techniques herein. The focus electronics and control system 170 may be used to perform sharpness calculations using the information from the dither focusing stage 150 and focus sensor 160. The sharpness values may be calculated over at least a portion of a sinusoidal curve defined by dither movement. The focus electronics and control system 170 may then use the information to determine the position for the best focus image of the tissue and command the slow focus stage 140 to move the microscope objective 120 to a desired position (along the Z-axis, as shown) for obtaining the best focus image during the imaging process. The control system 170 may also use the information to control the speed of the XY moving stage 130, for example, the speed of movement of the stage 130 in the Y direction. In an embodiment, sharpness values may be computed by differencing contrast values of neighboring pixels, squaring them and summing those values together to form one score. Various algorithms for determining sharpness values are further discussed elsewhere herein.

In various embodiments according to the system described herein, and in accordance with components discussed elsewhere herein, a device for creating a digital image of a specimen on a microscope slide may include: a microscope objective that is infinity corrected; a beam splitter; a camera focusing lens; a high-resolution camera; a sensor focus lens group; a dither focusing stage; a focusing sensor; a focusing coarse (slow) stage; and focus electronics. The device may allow for focusing the objective and capturing each snapshot through the camera without the need for predetermining a focus point for all snapshots prior to capturing the snapshots, and wherein the total time for capturing all the snapshots is less than the time required by a system requiring a step of predetermining focus points for each snapshot prior to capturing the snapshots. The system may include computer controls for: i) determining a first focus point or a few focus points, herein called pre-scan, anchor or definite tissue points, on the tissue to establish a nominal focus plane by moving the coarse focus stage through the entire z range and monitoring sharpness values; ii) positioning the tissue in x and y to start at a corner of an area of interest; iii) setting the dither fine focus stage to move, wherein the dither focus stage is synchronized to a master clock which also controls the velocity of the xy stage; iv) commanding the stage to move from frame to adjacent frame, and/or v) producing a trigger signal to acquire a frame on the image sensor and trigger a light source to create a pulse of light.

Further, according to another embodiment, the system described herein may provide computer-implemented method for creating a digital image of a specimen that has been deposited on a microscope slide. The method may include determining a scan area comprising a region of the microscope slide that includes at least a portion of the specimen. The scan area may be divided into a plurality snapshots. The snapshots may be captured using a microscope objective and a camera, in which focusing the objective and microscope and capturing each snapshot through the camera may be conducted for each snapshot without the need for predetermining a focus point for all snapshots prior to capturing the snapshots. The total time for capturing all the snapshots may be less than the time required by a method requiring a step of predetermining focus points for each snapshot prior to capturing the snapshots.

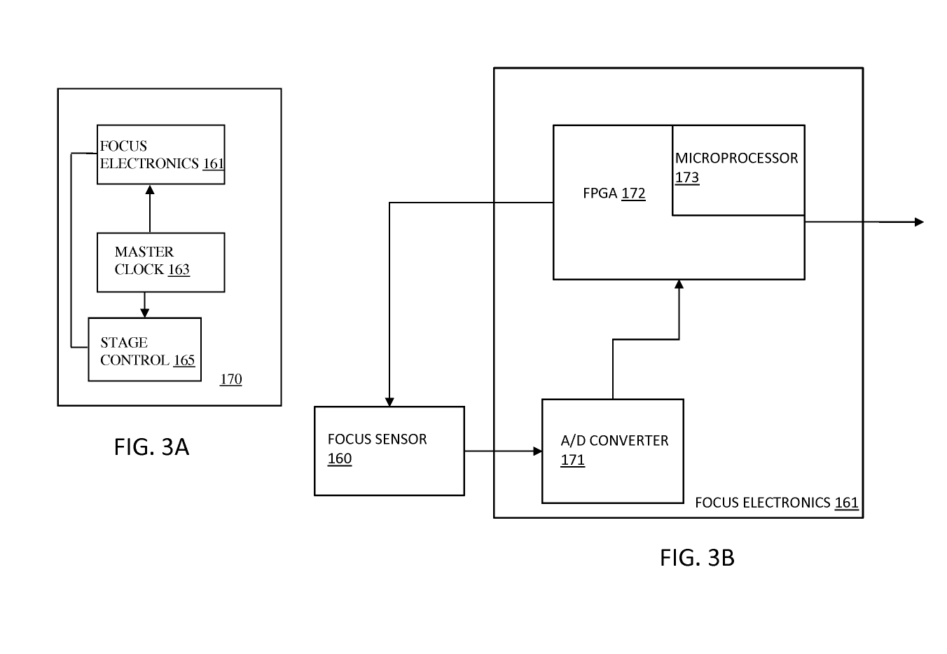

FIG. 3A is a schematic illustration of an embodiment of the focus electronics and control system 170 including focus electronics 161, a master clock 163 and stage control electronics 165. FIG. 3B is a schematic illustration of an embodiment of the focus electronics 161. In the illustrated embodiment, the focus electronics 161 may include appropriate electronics such as a suitably fast A/D converter 171 and a field-programmable gate array (FPGA) 172 with a microprocessor 173 that may be used to make sharpness calculations and/or perform other processing as further discussed elsewhere herein. The A/D converter 171 may receive information from the focus sensor 160 which is coupled to the FPGA 172 and microprocessor 173 and used to output sharpness information. The master clock included in 170 may supply the master clock signal to the focus electronics 161, stage control electronics 165, and other components of the system. The stage control electronics 165 may generate control signals used to control the slow focus stage 140, X-Y moving stage 130, dither focusing stage 150, and/or other control signals and information, as further discussed elsewhere herein. The FPGA 172 may supply a clock signal to the focus sensor 160, among other information. Measurements in the lab show a sharpness calculation on a 640×32 pixel frame can be made in 18 microseconds, easily fast enough for suitable operation of the system described herein. In an embodiment, the focus sensor 160 may include a monochrome CCD camera windowed to 640×32 strip, as further discussed elsewhere herein.

The scanning microscope may acquire either a 1D or 2D array of pixels including contrast information, and/or intensity information in RGB or some other color space as further discussed elsewhere herein. The system finds best focus points over a large field, for example on a glass slide 25 mm×50 mm. Many commercial systems sample the scene produced by a 20×, 0.75 NA microscope objective with a CCD array. Given the NA of the objective and condenser of 0.75 and wavelength of 500 nm the lateral resolution of the optical system is about 0.5 micron. To sample this resolution element at the Nyquist frequency, the pixel size at the object is about 0.25 micron. For a 4 Mpixel camera (e.g., a Dalsa Falcon 4M30/60), running at 30 fps, with a pixel size of 7.4 micron the magnification from the object to the imaging camera is 7.4/0.25=30×. The system described herein is desirably used where tissue spatial variation in the focus dimension is much lower than the frame size at the object. Variations in focus, in practice, occur over greater distances and most of the focus adjustment is made to correct for tilts. These tilts are generally in the range of 0.5-1 micron per frame dimension at the object.

Time to result for current scanning systems (e.g., a Biolmagene iScan Coreo system) is about 3.5 minutes for pre-scan and scan of a 20×15 mm×15 mm field and about 15 minutes for a 40× scan on 15 mm×15 mm field. The 15 mm×15 mm field is scanned by running 35 frames in 26 passes. The scans may be done uni-directionally with a 1 sec retrace time. The time to scan using a technique according to the system described herein may be about 5 seconds to find the nominal focus plane, 1.17 seconds per pass (25 passes), for a total of 5+25×(1.17+1)=59.25 seconds (about 1 minute). This is a considerable time savings over conventional approaches. Other embodiments of the systems described herein may allow even faster focus times, but a limitation may occur on the amount of light needed for short illumination times to avoid motion blur on continuous scan. Pulsing or strobing the light source 118, which may be an LED light source as further discussed elsewhere herein, to allow high peak illumination can mitigate this issue. In an embodiment, the pulsing of the light source 118 may be controlled by the focus electronics and control system 170. In addition, running the system bi-directionally would eliminate the retrace time saving about 25 seconds for a 20× scan resulting in a scan time of 35 seconds.

It should be noted that the components used in connection with the focus electronics and control system 170 may also more generally be referred to as electrical components used to perform a variety of different functions in connection with embodiments of the techniques described herein.

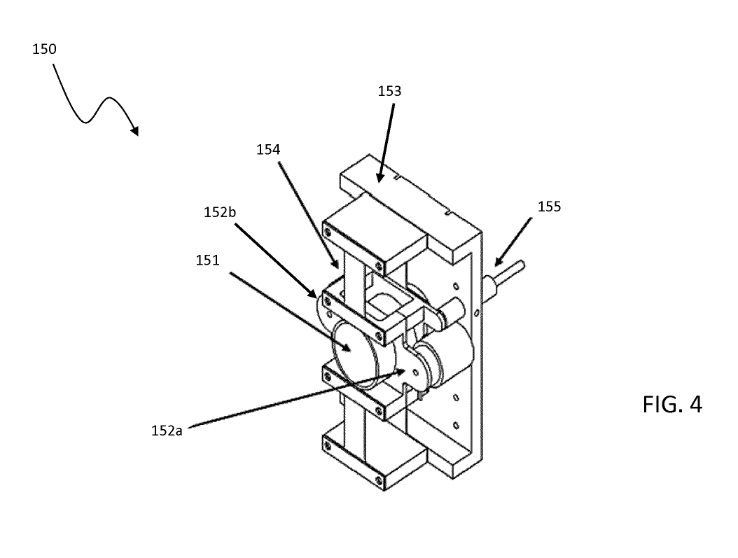

FIG. 4 is a schematic illustration showing the dither focus stage 150 in more detail according to an embodiment of the system described herein. The dither focus stage 150 may include a dither focusing lens 151 that may be moved by one or more actuators 152a,b, such as voice coil actuators, and which may be mounted into a rigid housing 153. In an embodiment, the lens may be achromatic lens having a 50 mm focal length, as is commercially available, see for example Edmund Scientific, NT32-323. Alternatively, the dither focusing lens 151 may be constructed from plastic, aspheric and shaped such that the weight of the lens is reduced (extremely low-mass). A flexure structure 154 may be attached to the rigid housing 153 and attached to a rigid ground point and may allow only translational motion of the dither focusing lens 151, for example, small distances of about 600-1000 microns. In an embodiment, the flexure structure 154 may be constructed of an appropriate stainless steel sheets, of about 0.010″ thick in the bending direction and form a four-bar linkage. The flexure 154 may be designed from a suitable spring steel at a working stress far from its fatigue limit (factor of 5 below) to operate over many cycles.

The moving mass of the dither focusing lens 151 and flexure 154 may be designed to provide about a 60 Hz or more first mechanical resonance. The moving mass may be monitored with a suitable high bandwidth (e.g., >1 kHz) position sensor 155, such as a capacitive sensor or eddy current sensor, to provide feedback to the control system 170 (see FIG. 2). For example, KLA Tencor's ADE division manufactures a capacitive sensor 5 mm 2805 probe with a 1 kHz bandwidth, 1 mm measurement range, and 77 nanometer resolution suitable for this application. The dither focus and control system, such as represented by functionality included in element 170, may keep the amplitude of the dither focusing lens 151 to a prescribed focus range. The dither focus and control system may rely on well-known gain-controlled oscillator circuits. When operated in resonance the dither focusing lens 151 may be driven at low current, dissipating low power in the voice coil windings. For example, using a BEI Kimco LAO8-10 (Winding A) actuator the average currents may be less than 180 mA and power dissipated may be less than 0.1 W.

It is noted that other types of motion of the dither lens and other types of actuators 152a,b may be used in connection with various embodiments of the system described herein. For example, piezoelectric actuators may be used as the actuators 152a,b. Further, the motion of the dither lens may be motion at other than resonant frequencies that remains independent of the motion of the microscope objective 120.

The sensor 155, such as the capacitive sensor noted above and which may be included in an embodiment in accordance with techniques herein, may provide feedback as to where the dither focusing lens is positioned (e.g. with respect to the sine wave or cycle corresponding to the movements of the lens). As will be described elsewhere herein, a determination may be made as to which image frame obtained using the focus sensor produces the best sharpness value. For this frame, the position of the dither focusing lens may be determined with respect to the sine wave position as indicated by the sensor 155. The position as indicated by the sensor 155 may be used by the control electronics of 170 to determine an appropriate adjustment for the slow focusing stage 140. For example, in one embodiment, the movement of the microscope objective 120 may be controlled by a slow stepper motor of the slow focus stage 140. The position indicated by the sensor 155 may be used to determine a corresponding amount of movement (and corresponding control signal(s)) to position the microscope objective 120 at a best focus position in the Z direction. The control signal(s) may be transmitted to the stepper motor of the slow focus stage 140 to cause any necessary repositioning of the microscope objective 120 at the best focus position.

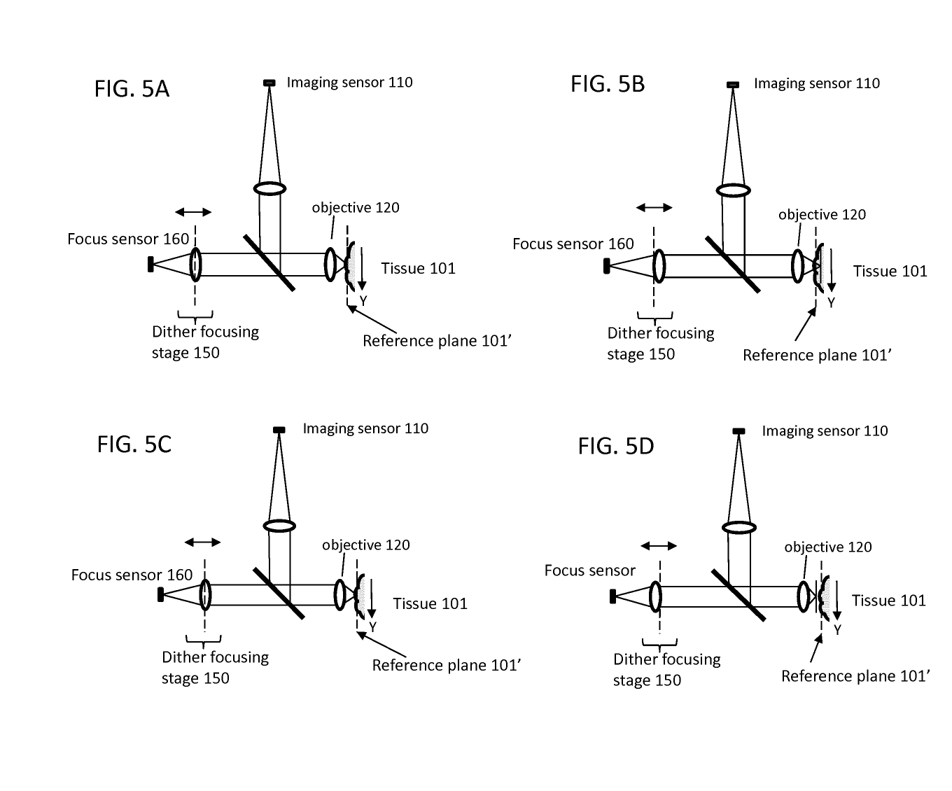

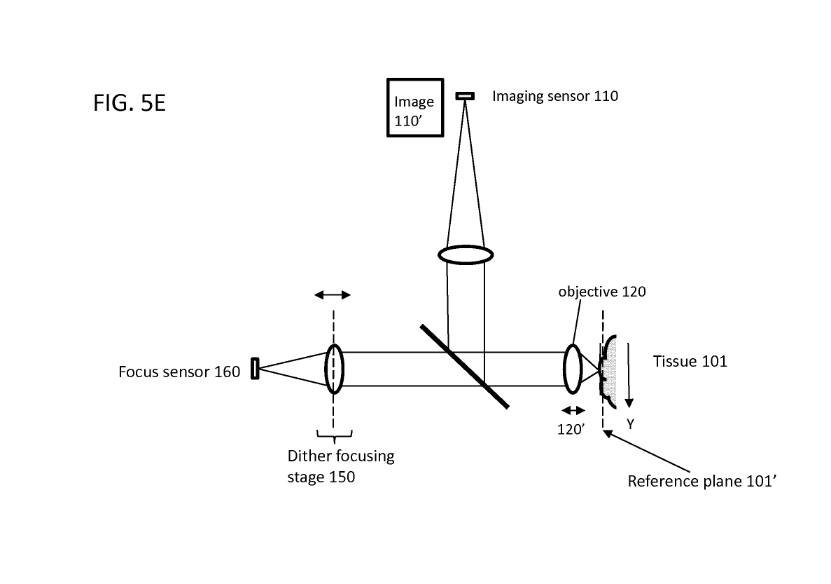

FIGS. 5A-5E are schematic illustrations showing an iteration of the focusing operations according to the system described herein. The figures show the image sensor 110, the focus sensor 160, the dither focusing stage 150 with a dither lens and the microscope objective 120. The tissue 101 is illustrated moving in the y-axis, i.e. on the XY moving stage 130, while the focus operations are performed. In an example, the dither focusing stage 150 may move the dither lens at a desired frequency, such as 60 Hz or more (e.g., 80 Hz, 100 Hz), although it is noted that, in other embodiments, the system described herein may also operate with the dither lens moving at a lower frequency (e.g., 50 Hz) according to applicable circumstances. The XY moving stage 130 may be commanded to move, e.g., in the Y direction, from frame to adjacent frame. For example, the stage 130 may be commanded to move at a constant of 13 mm/sec which for a 20× objective corresponds to an acquisition rate of about 30 frames/sec. Since the dither focus stage 150 and XY moving stage 130 may be phase locked, the dither focus stage 150 and sensor 160 may make 60 focus calculations per second, or functioning bi-directionally (reading on the up and down motion of the sine wave) 120 focus points per second or 4 focus points per frame. For a frame height of 1728 pixels, this equates to a focus point every 432 pixels or for the 20× objective every 108 microns. Since the XY moving stage 130 is moving, the focus point should be captured in a very short period of time, for example 330 μsec (or less), to keep the variation in the scene minimal.

In various embodiments, as further discussed elsewhere herein, this data may be stored and used to extrapolate the next frame's focus position or, alternatively, extrapolation may not be used and the last focus point is used for the focus position of the active frame. With a dither frequency of 60 Hz and a frame rate of 30 frames per second the focus point is taken at a position no more than ¼ of a frame from the center of the snapped frame. Generally, tissue heights do not change enough in ¼ of a frame to make this focus point inaccurate.

A first focus point may be found on the tissue to establish the nominal focus plane or reference plane 101′. For example, the reference plane 101′ may be determined by initially moving the microscope objective 120, using the slow focus stage 140, through the entire Z range, e.g., +1/−1 mm, and monitoring sharpness values. Once the reference plane 101′ is found, the tissue 101 may be positioned in X and Y to start at a corner, and/or other particular location, of the area of interest, and the dither focusing stage 150 is set to move, and/or otherwise movement of the dither focusing stage 150 continues to be monitored, beginning in FIG. 5A.

The dither focus stage 150 may be synchronized to a master clock in the control system 170 (see FIG. 2) which may also be used in connection with controlling the velocity of the XY moving stage 130. For example, if the dither focus stage 150 were to move through a 0.6 millimeter p-v (peak to valley) sinusoidal motion at 60 Hertz, assuming an 32% duty cycle to use the sinusoid's more linear range, 8 points could be collected through the focus range over an 2.7 msec period. In FIGS. 5B-5D the dither focusing stage 150 moves the dither lens in a sinusoidal motion and focus samples are taken along through at least a portion of the sinusoidal curve. Focus samples would be taken therefore every 330 μsec or at a rate of 3 kHz. With a magnification of 5.5× between the object and the focus sensor 160, a motion at the dither lens of 0.6 mm p-v equates to a 20 micron p-v motion at the objective lens. This information is used to convey the position at which highest sharpness is computed, i.e. the best focus, to the slower stepper motor of the slow focus stage 140. As shown in FIG. 5E, the slow focus stage 140 is commanded to move the microscope objective 120 to the best focus position (illustrated by motion range 120′) in time for the image sensor 110 to capture the best focus image 110′ of the area of interest of the tissue 101. In an embodiment, the image sensor 110 may be triggered, e.g. by the control system 170, to snapshot an image after a specific number of cycles of the dither lens motion. The XY moving stage 130 moves to the next frame, the cyclical motion of the dither lens in the dither focus stage 150 continues, and focusing operations of FIGS. 5A-5E are repeated. Sharpness values may be calculated at a rate that does not bottleneck the process, e.g., 3 kHz.

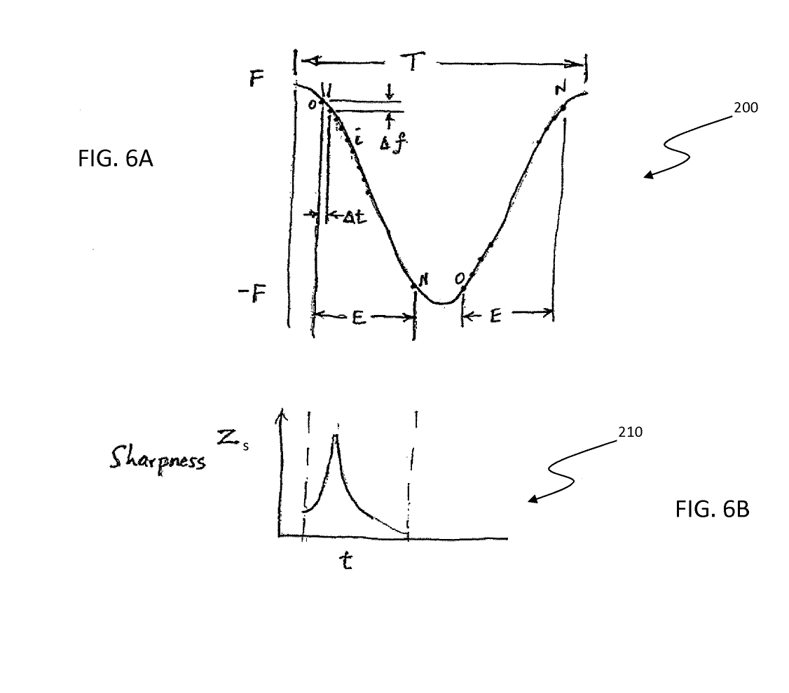

FIG. 6A is a schematic illustration of a plot 200 showing the command waveform of the dither focus optics and sharpness determinations according to an embodiment of the system described herein. In an embodiment based on the times discussed in connection with the example of FIGS. 5A-5E:

T=16.67 msec, /*period of the dither lens sinusoid if the lens resonates at 60 Hz */
F=300 μm, /* positive range of focus values */
N=8, /* number of focus points obtained in the period E */
Δt=330 μsec, /* focus point samples obtained every 330 μsec */
E=2.67 msec, /* the period over which the N focus points are obtained */
Δf=1.06 μm at center of focus travel. /* step size of focus curve */

Therefore with this duty cycle of 32%, 8.48 μm (8×1.06 μm=8.48 μm) is sampled through focus processing.

FIG. 6B is a schematic illustration showing a plot 210 of calculated sharpness (Zₛ) values for a portion of the sine wave motion of the dither lens shown in the plot 210. The position (z) for each focus plane sampled as a function of each point i is given by EQUATION 1:

$$z = F cos [ 2 π [ \frac{( T - 2 E )}{4} + Δ t \cdot i ] \frac{1}{T} ] \qquad EQUATION 1$$

Windowing down a CCD camera may provide a high frame rate suitable for the system described herein. For example, the company Dalsa of Waterloo, Ontario, Canada produces the Genie M640-1/3 640×480 Monochrome camera. The Genie M640-1/3 will operate at 3,000 frame/sec at a frame size of 640×32. The pixel size on the CCD array is 7.4 microns. At the 5.5× magnification between the object and focus plane, one focus pixel is equivalent to about 1.3 micron at the object. Though some averaging of about 16 object pixels (4×4) per focus pixel may occur, sufficient high spatial frequency contrast change is preserved to obtain good focus information. In an embodiment, the best focus position may be determined according to the peak value of the sharpness calculations plot 210. In additional embodiments, it is noted that other focus calculations and techniques may be used to determine the best focus position according to other metrics, including the use of a contrast metric, as further discussed elsewhere herein.

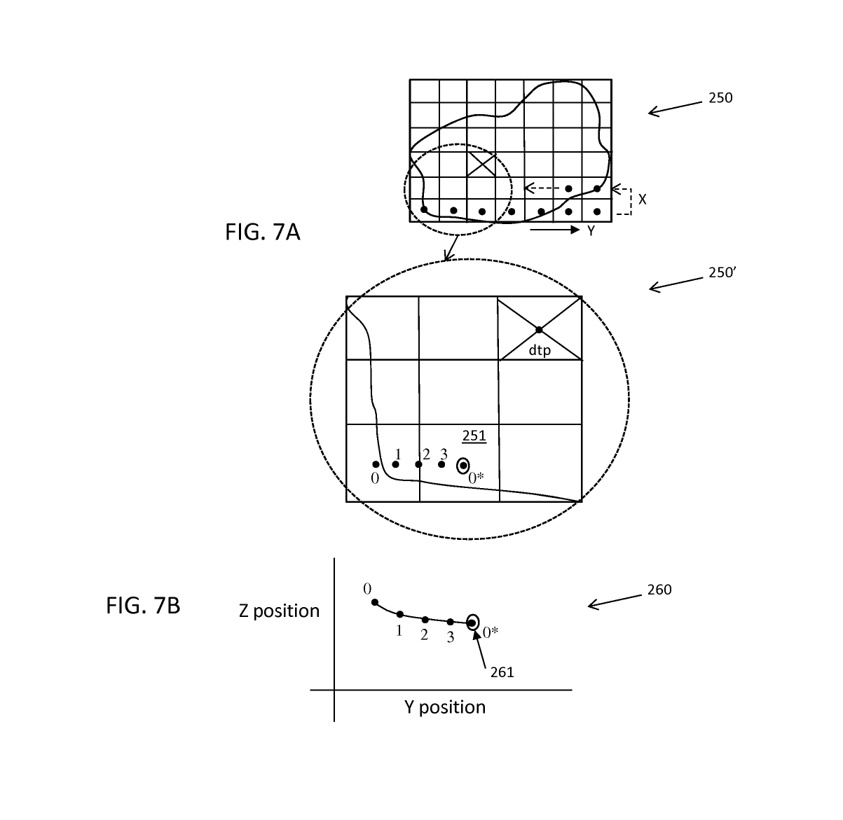

FIGS. 7A and 7B are schematic illustrations showing focusing determinations and adjustments of a specimen (tissue) according to an embodiment of the system described herein. In FIG. 7A, illustration 250 is a view of the specimen shown in approximate image frames in connection with movement of the specimen along the Y-axis according to movement of the XY moving stage 130 discussed herein. One traversal or pass over the specimen in connection with movement of the specimen along the Y-axis and X-axis (e.g., according to movement of the XY stage) is illustrated in 250, illustrating a serpentine pattern for traversing the specimen. Illustration 250′ is an enlarged version of one portion of the illustration 250. One frame of the illustration 250′ is designated dtp, referring to a definite tissue point or anchor point of the specimen. In the example of illustration 250′, a specimen boundary is shown and, during the scan thereover, multiple focus calculations are performed in accordance with the system described herein. In the frame 251, and by way example, there is illustrated that a best focus determination is made after 4 focus calculations (shown as focus positions 1, 2, 3 and 0*) are performed in connection with imaging the specimen, although more focus calculations may be performed in connection with the system described herein. FIG. 7B shows a schematic illustration 260 showing a plot of the Z-axis position of the microscope objective in relation to Y-axis position of the specimen being examined. The illustrated position 261 shows the determined position along the Z-axis for adjusting the microscope objective 120 to achieve best focus according to an embodiment of the system described herein.

It should be noted that the system described herein provides significant advantages over conventional systems, such as those described in U.S. Pat. Nos. 7,576,307 and 7,518,642, which are incorporated herein by reference, in which the entire microscope objective is moved through focus in a sinusoid or triangular pattern. The system provided herein is advantageous in that it is suitable for use with microscope objective and an accompanying stage that are heavy (especially if other objectives are added via a turret) and cannot be moved at the higher frequencies described using the dither optics. The dither lens described herein may have an adjusted mass (e.g., be made lighter, less glass) and the imaging demands on the focus sensor are less than that imposed by the microscope objective. The focus data may be taken at high rates, as described herein, to minimize scene variation when computing sharpness. By minimizing scene variation, the system described herein reduces discontinuities in the sharpness metric as the system moves in and out of focus while the tissue is moving under the microscope objective. In conventional systems, such discontinuities add noise to the best focus calculation.

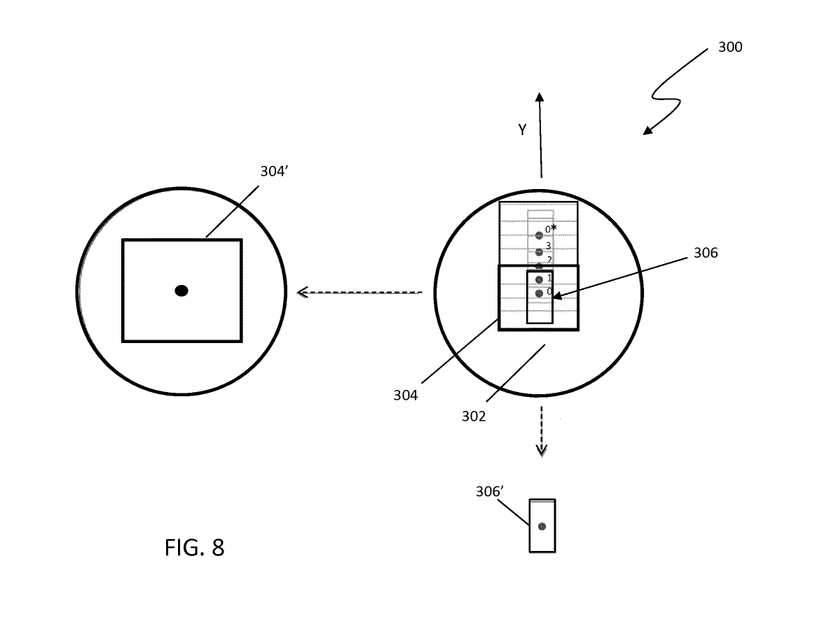

FIG. 8 is a schematic illustration 300 showing a camera window 302 including an image frame 304 of an image sensor and a focus frame 306 of a focus sensor. The field of view of each of the focus frame 306 and the image frame 304 are shown aligned. The image frame 304 may be oriented in the direction of travel of the stage 130, such that a column of frames acquired during imaging is aligned with the camera window 302. The field of view in the image frame 304, using, e.g., a Dalsa 4M30/60 CCD camera, 2352×1728 pixels, 7.4 micron square pixel, is 0.823 mm×0.604 mm using a 21× magnification tube lens. The image frame's wider dimension (0.823 mm) may be oriented perpendicular to the longer dimension of the focus frame 306. The focus frame 306 of the focus sensor (e.g., Dalsa Genie 640×480 pixels, 7.4 micron square pixel) may be windowed to a rectangle 306′ of 100 pixels by 320 pixels or 0.148 mm×0.474 mm at the object using a 5× magnification in the focus leg. The focus frame 306 therefore sees much of the tissue seen by the image frame 304. This increases the probability of capturing tissue in a focus operation even if the tissue sections are sparsely distributed within the frame. The large area of the tissue viewed by the focus frame 306 provides for less noise, and higher sensitivity, in determining best focus and may be advantageously used in discriminating between non-tissue and tissue areas. According to an embodiment of the system described herein, 60 best focus determinations may be made per second, with 20 sharpnesses calculated for each focus sensor cycle, resulting in 1200 sharpness calculations per second for a 60 Hz focus dither. Focus calculations (e.g., focus positions 1, 2, 3 and 0* as described in FIGS. 7A and 7B) are performed in connection with imaging the specimen. A best focus image frame is shown as image frame 304′. Coverage of the tissue is established by executing a serpentine pattern traversing the complete area of interest.

An example of the sharpness computation is shown in EQUATION 2 (e.g., based on use of a camera windowed to a 320×100 area). For row i, dimension n up to 100, and column j, dimension m up to 320/z, where z is the number of zones for which sharpness is calculated, sharpness for a zone may be represented by EQUATION 2:

Sharpness=Σᵢ₌₀ⁿ⁻¹Σⱼ₊₀^{m-k-1}(1_{i,j}−1_{i,j+k})²Σⱼ₊₀^{m-k-1}[(I_{i,j}−I_{i,j+k})²] EQUATION 2

where k is an integer between or equal to 1 and 5. For this embodiment, z=1 (only one zone), although, in other embodiments, as further discussed elsewhere herein, more than one zone may be used in connection with the system described herein. Other sharpness metrics and algorithms may also be used in connection with the system described herein. As the XY moving stage 130 is moving along the y-axis, the system acquires sharpness information for the current zone in the focus frame 306, which information is used to determine a best focus position.

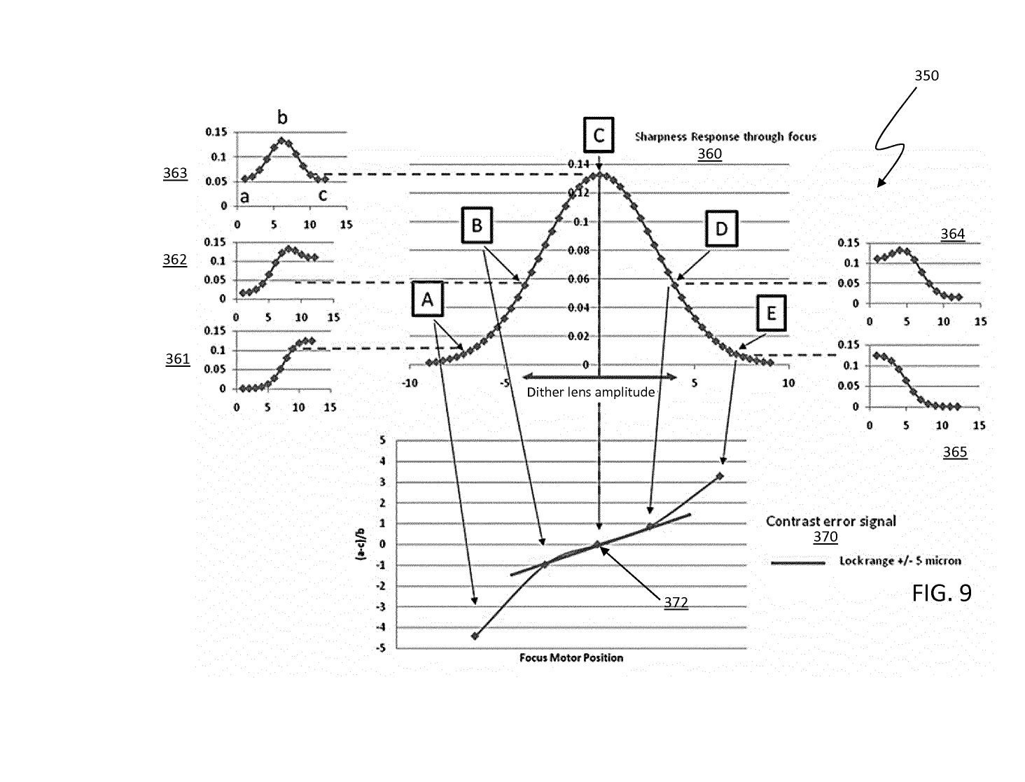

FIG. 9 is a schematic illustration 350 showing an example of a sharpness profile, produced from moving through focus positions, including a sharpness response curve 360 and contrast error signal 370 for each sharpness response at multiple points that are sampled by the dither focusing optics according to an embodiment of the system described herein. Plot 360 shows dither lens amplitude in micrometers in the x-axis and sharpness units along the y-axis. As illustrated, the dither lens motion may be centered at representative positions A, B, C, D and E; however, is it noted that the computations described herein may be applied to each of the points on the sharpness curve. The sharpness response produced from the focus sensor 160, for a half cycle of the dither lens sinusoid, when motion of the dither lens is centered at each of the positions A, B, C, D and E is shown, respectively, in the waveform plots 361-365.

As discussed herein, the dither lens may be vibrated at 60 Hz at approximately 300 microns peak-to-peak (p-t-p) amplitude. This produces a change in focus as seen by the focus sensor of about +/−5 microns at the tissue. Best focus can be measured by the focus sensor by computing sharpness at each focus frame. This calculation may be done in the camera's FPGA. Therefore while the dither lens is vibrating at 60 Hz, 20 sharpness metrics may be computed per dither cycle (1200 sharpness calculations per second). Characteristic waveforms 361-365 are measured depending on the position of the microscope objective relative to best focus. For example, at best focus (position C) the dither lens samples either side of the sharpness response and produces sine wave (waveform 363) at two times the frequency of the dither vibration. Point ‘a’ at a sine wave trough, point ‘b’ at the peak and point ‘c’ at the subsequent trough can be used to compute an error signal to be used to control focus, for example, by controlling the slow focus stage 140 to move the microscope objective 120 into the best focus position before the image sensor 110 captures the image 110′. Points a, b and c are sharpness values, from waveforms 361-365, obtained in connection with each centered point (e.g., A, B, C, D, E) of the dither lens motion shown on the sharpness response curve 360 for computing a contrast error signal 370.

In an embodiment, the Contrast Error Signal (CES) 370 may be an error function computed as shown by EQUATION 3:

CES=(a−c)/b. EQUATION 3

At positions off-focus, for example at position A (see waveform 361), CES is negative, moving to a smaller negative number at position, B (see waveform 362). CES becomes zero at position C (see waveform 363, for points a, b and c taken therefrom) and increasingly positive as the system moves away from focus through positions D and E (see waveforms 364 and 365). The point where CES is zero (position C) indicates the best focus position 372 for the focus motor. This CES error function can then be used in a feedback loop to control the slow focus motor, as further discussed elsewhere herein. Areas outside of the “lock range” of +/−5 microns have a characteristic frequency equal to the dither frequency. Moving further out of focus produces progressively smaller amplitude of the waveform. In areas of constant contrast or non-tissue areas, the amplitude of the waveform will be very small or provide a constant signal with no oscillation. Setting a threshold on the amplitude of the waveform can determine whether tissue is in view or not in view.

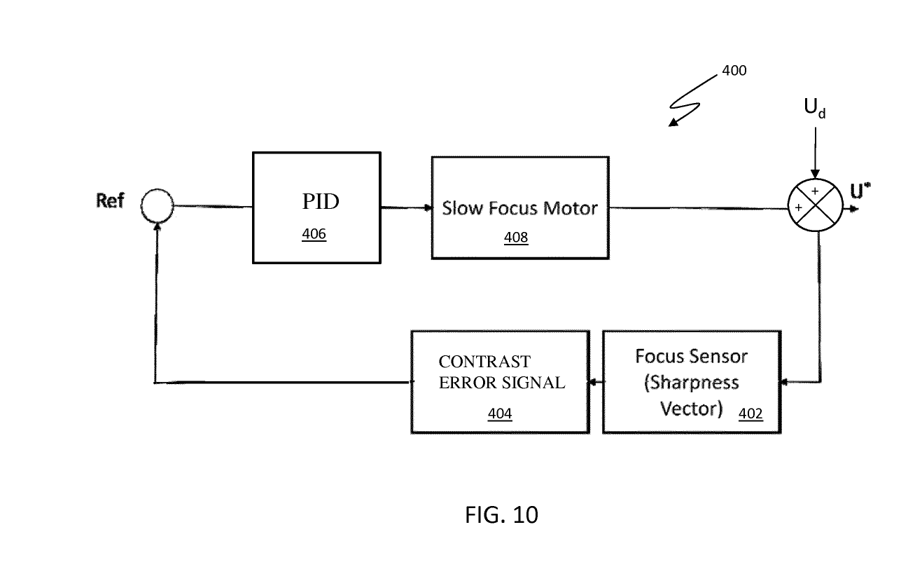

FIG. 10 shows a functional control loop block diagram 400 illustrating use of the contrast error signal to produce a control signal to control the slow focus stage 140. U_{d} may be considered as a disturbance to the focus control loop and may represent the slide tilt or changing tissue surface heights, for example. Functional block 402 shows generation of sharpness vector information that may be generated by the focus sensor 160 and communicated to the focus electronics and control system 170. Functional block 404 shows generation of a contrast number (e.g., value of the contrast error signal, such as by EQUATION 3) at the point the dither lens is sampling focus. This contrast number is compared to a set point or reference value (Ref) produced at an initial step where best focus was previously established.

A proportional (P), integrating (I), and differentiating (D) (PID) function block 406 uses corresponding known control theory techniques to correct the slow focus motor which acts (at functional block 408) to keep the scene in focus and provides optimal stability and response to a disturbance (such as a sudden change in focus). Based on an appropriate control loop response speed, the system can dynamically focus while acquiring a column of image data. It should be noted that an embodiment may adjust the position of the microscope objective 120 in accordance with a minimum or threshold amount of movement. Thus, such an embodiment may avoid making adjustments smaller than the threshold.

Alternatively, in another embodiment, the system may move the Y stage/slide in the Y direction to acquire focus data using the above approach and store the best focus position for the column. This can be done very rapidly due to the dither focus approach. The system can retrace the column imaging the scene using this focus data. The next column is scanned in the same way. Column scanning continues until the area of interest has been acquired.

Alternatively, in yet another embodiment, the first column of data may be used to update a best focus surface produced by sparse pre-scan data. For example, an area of interest is scanned in a serpentine pattern by imaging the first column, storing the best focus data produced by the above dither lens method, using that focus data to recomputed the best focus surface, then imaging the second column, etc. until the area of interest is scanned.

Alternatively, in yet another embodiment, the focus sensor can be aligned such as its field of view is entirely in an adjacent column. An area of interest is scanned in a serpentine pattern. The first column (Column 1) of data scanned simply stores the best focus data for the adjacent column (Column 2). On the return pass Column 2 is imaged using the best focus data and Column 3 best focus data is stored and so on until the entire area of interest is scanned.

The methods described herein provide for very fast scanning while providing more focus information to keep the tissue at best focus.

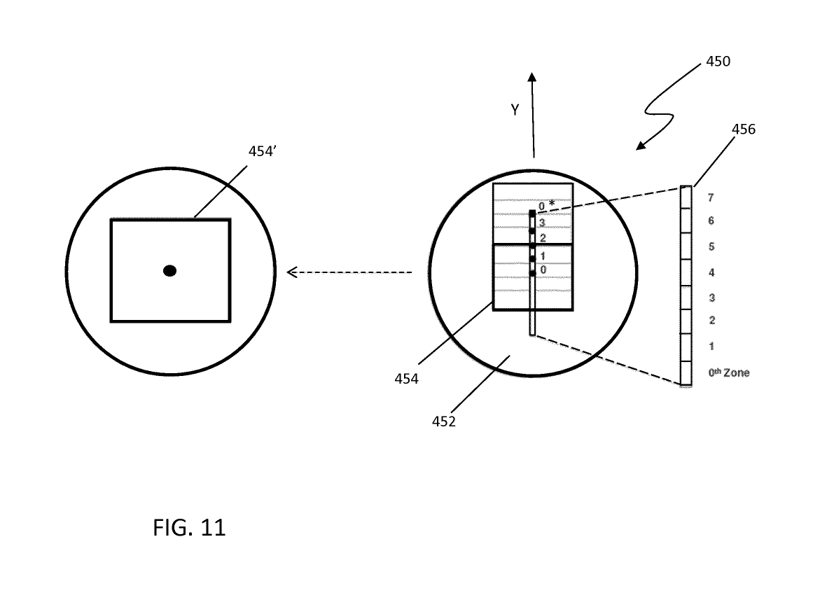

FIG. 11 is a schematic illustration 450 of a camera window 452 showing the focus window 456 being broken up into zones in connection with focus processing according to another embodiment of the system described herein. In the illustrated embodiment, the focus frame 456 is subdivided into 8 zones; however, fewer or more than 8 zones may be used in connection with the system described herein. A first subset of the zones may be within a snapshot n and a second subset of zones is within snapshot n+1. For example, Zones 2, 3, 4, 5 are within the image frame 454 snapped at time t1. Zones 6 and 7 may be completely within the next image frame to be snapped as the XY moving stage 130 traverses from bottom to top in the figure and/or Zones 0 and 1 may be completely within the next image frame to be snapped as the stage 130 traverses from top to bottom of the figure. Focus positions 0, 1, 2, and 3 may be used to extrapolate the best focus position for the next snapped frame at position 0*. Coverage of the tissue may be established, for example, by executing a serpentine pattern traversing the complete area of interest. The image frame's 406 wider dimension may be oriented perpendicular to the longer dimension of the focus frame 456 and allows the minimum number of columns traversed over a section of tissue. In various embodiments, the focus frame 456 of the focus sensor may be longer, in various ranges, than the image frame 404 of the image sensor, and may be advantageously used in connection with a look-ahead focusing technique involving multiple zones, as further discussed elsewhere herein. When computing a sharpness metric for a single focus point using multiple zones, the sharpness metric may be determined for each zone and combined, for example, such as by adding all sharpness metrics for all zones considered at such a single point. The best focus image is shown in frame 454′.

During the scanning process, it may be advantageous to determine whether the system is transitioning from a white space (no tissue) to a darker space (tissue). As the XY moving stage 130 is moving along the y-axis, the system acquires sharpness information for all of the Zones 0-7 in the focus window 402. It is desirable as the stage 130 is moving to know how the tissue section heights are varying. By computing sharpness, in Zones 6 and 7, for example, it is possible to predict if this transition is about to occur. While scanning the column, if Zones 6 and 7 show increased sharpness, the XY moving stage 130 may be commanded to slow down to create more closely spaced focus points on the tissue boundary. If on the other hand a movement from high sharpness to low sharpness is detected, then it may be determined that the scanner view is entering a white space, and it may be desirable to slow down the stage 130 to create more closely spaced focus points on the tissue boundary. In areas where these transitions do not occur, the stage 130 may be commanded to move at higher constant speeds to increase the total throughput of slide scanning Sharpness calculations made be made as discussed in connection with EQUATION 2, and, in this embodiment, based on use of a camera windowed to a 640×32 strip. For example, row i, dimension n may be up to 32, and column j, dimension m may be up to 640/z, where z is the number of zones (e.g., 8 zones; Zones 0-7). This method may allow for advantageously fast scanning tissue. According to the system described herein, snapshots may be taken while focusing data is collected. Furthermore, all focus data may be collected in a first scan and stored and snapshots may be taken at best focus points during a subsequent scan. An embodiment may use contrast function values in a manner similar to that as described herein with sharpness values to detect changes in focus and accordingly determine transitions into, or out, of areas containing tissue or white space.

In another embodiment, a color camera may be used as the focus sensor 160 and a chroma metric may be determined alternatively and/or additionally to the sharpness contrast metric. For example, a Dalsa color version of the 640×480 Genie camera may be suitably used as the focus sensor 160 according to this embodiment. The chroma metric may be described as colorfulness relative to the brightness of a similarly illuminated white. In equation form (EQUATIONS 4A and 4B), chroma (C) may be a linear combination of R, G, B color measures:

C_{B}=−37.797×R−74.203×G+112×B EQUATION 4A

C_{R}=112×R−93.786×G−18.214×B EQUATION 4B

Note for R=G=B, C_{B}=C_{R}=0. A value for C, representing total chroma, may be determined based on C_{B} and C_{R}. (e.g., such as by adding C_{B} and C_{R}).

As the XY moving stage 130 is moving along the y axis, the focus sensor 160 may acquire color (R, G, B) information, as in a bright field microscope. The use of RGB color information may be used, as with the contrast technique, to determine whether the system is transitioning from a white space (no tissue) to a colorful space (tissue). In an embodiment, information concerning transitioning form a white space to a colorful space may be made in accordance with the processing of a focus frame, having a field of view substantially as large as the image frame field of view, and using only one zone as discussed in connection with the illustration 300.

In another embodiment, look-ahead processing techniques may be used in connection with the system described herein. By computing chroma in Zones 6 and 7, for example, it is possible to predict if a transition between white space (no tissue) and colorful space (tissue) is about to occur. If, for example, very little chroma is detected, then C=0 and it may be recognized that no tissue boundaries are approaching. However, while scanning the focus column, if Zones 6 and 7 show increased chroma, then the stage 130 may be commanded to slow down to create more closely spaced focus points on the tissue boundary. If on the other hand a movement from high chroma to low chroma is detected, then it may be determined that the scanner is entering a white space, and it may be desirable to slow down the stage 130 to create more closely spaced focus points on the tissue boundary. In areas where these transitions do not occur, the stage 130 may be commanded to move at higher constant speeds to increase the total throughput of slide scanning

In connection with use of sharpness values, contrast ratio values, and/or chroma values to determine when the field of view or upcoming frame(s) is entering or exiting a slide area with tissue, processing variations may be made. For example, when entering an area with tissue from white space (e.g., between tissue areas), movement in the Y direction may be decreased and a number of focus points obtained may also increase. When viewing white space or an area between tissue samples, movement in the Y direction may be increased and fewer focus points determined until movement over an area containing tissue is detected (e.g., such as by increased chroma and/or sharpness values). It is noted that embodiments discussed herein may be configured for use with the look-ahead technique and/or may be configured for use with only one zone without using look-ahead processing. For example, a wider rectangular focus frame may be more suitable for focus processing using only one zone, while a longer strip-like focus frame, that may extend beyond the image frame, may be more suitable for use with look-ahead focus processing techniques.

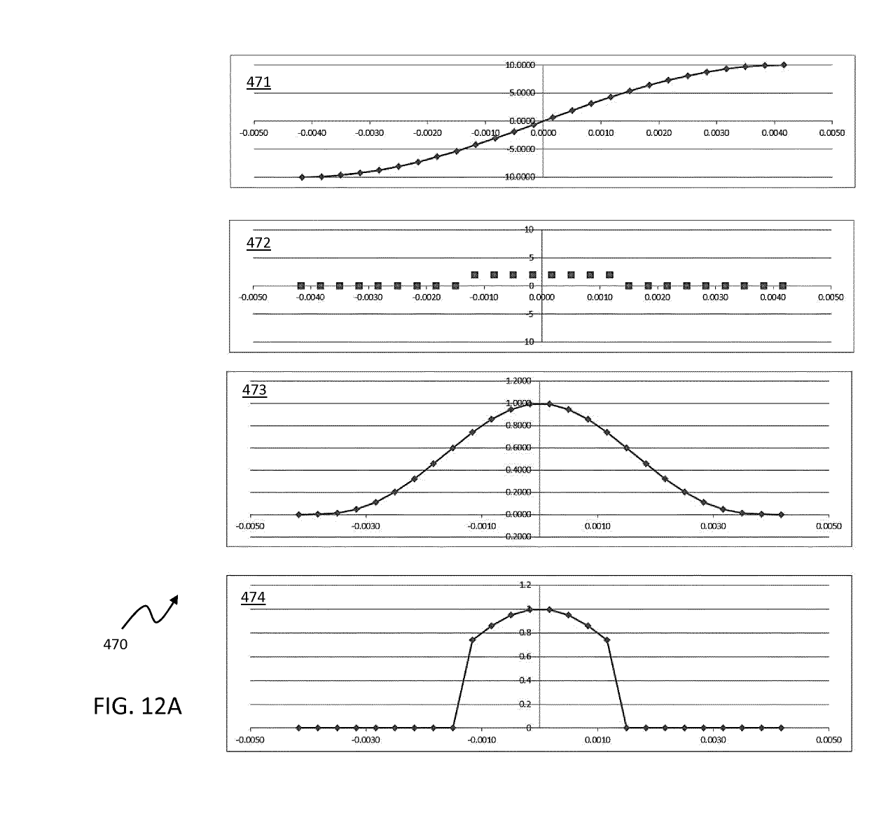

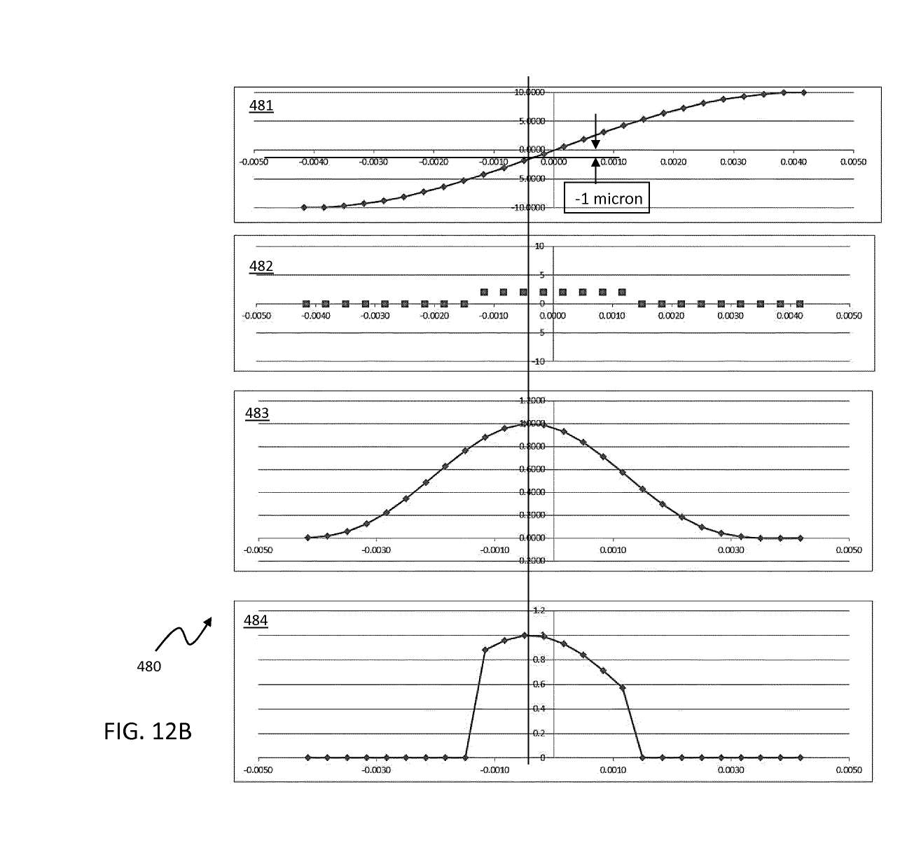

FIGS. 12A and 12B show graphical illustrations 470, 480 of plots in connection with focus techniques using sharpness values that may be obtained at points in time in accordance with embodiments of the system described herein.

FIG. 12A shows the plots illustration 470 for a system as described herein in which the system is currently in focus and no correction is needed. The top plot 471, plotting microns versus time in seconds, shows the dither lens position as a curve corresponding to a half sine wave cycle (e.g., half of a single peak to peak cycle or period) of the dither lens movement. Plot 472 shows a sampling clock over the linear region of the dither sine wave motion in which sampling occurs for clock values of 1. Plot 473 shows the sharpness (in arbitrary units) calculated from the sharpness metric using the set of sharpness values obtained as if every point was sampled by linearly moving through focus (in the z direction). Plot 474 shows the sharpness curve sampled over the linear region of the dither sine wave motion. The best focus z position is interpolated from sampled sharpness data. It is seen that, in this case, the system is in focus and no correction is needed; that is, peak sharpness corresponds to the shown dither lens position at the zero position around which the sharpness response is being computed (see, e.g. waveform 363 for position C in FIG. 9).

FIG. 12B shows the plots illustration 480 for a system as described herein in which the system is not in focus and focus correction is needed. The top plot 481, plotting microns versus time in seconds, shows the dither lens position as a curve corresponding to a half sine wave cycle (e.g., half of a single peak to peak cycle or period) of the dither lens movement. Plot 482 shows a sampling clock over the linear region of the dither sine wave motion in which sampling occurs for clock values of 1. Plot 483 shows the sharpness (in arbitrary units) calculated from the sharpness metric using the set of sharpness values obtained as if every point was sampled by linearly moving through focus (in the z direction). Plot 484 shows the sharpness curve sampled over the linear region of the dither sine wave motion. The best focus z position is interpolated from sampled sharpness data. It is seen that, in this case, the system needs focus correction in accordance with the techniques discussed herein; that is, peak sharpness is found at about −1 microns from the dither lens position (see, e.g., waveform 362 for position B in FIG. 9). As discussed herein, an error correction signal may be determined according to the techniques herein and correction information may be fed to the slow focus motor to keep the scene in focus.

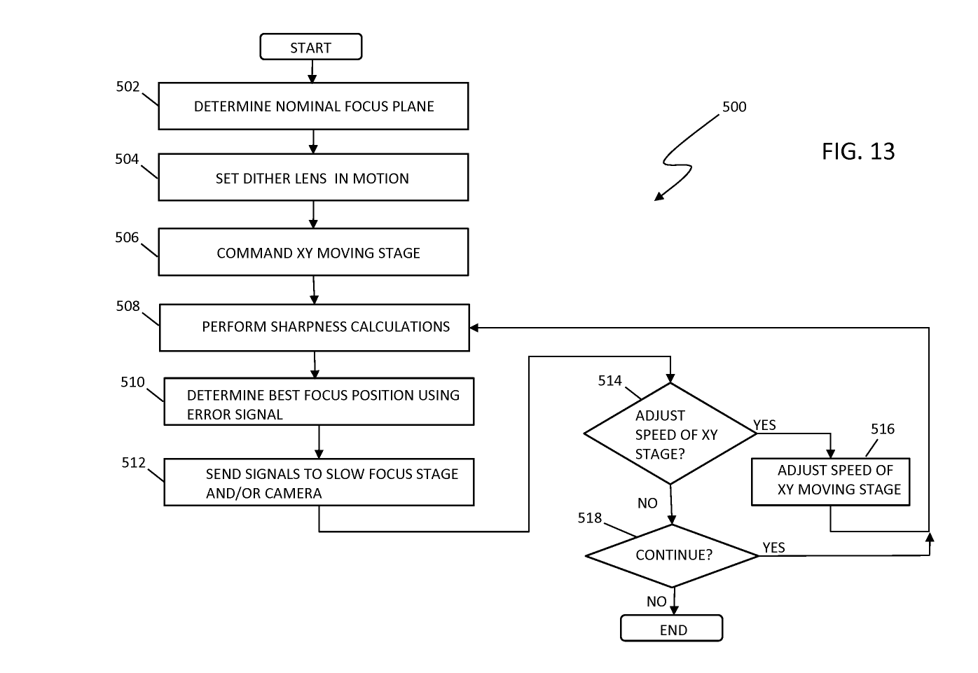

FIG. 13 is a flow diagram 500 showing on-the-fly focus processing during scanning of a specimen under examination according to an embodiment of the system described herein. At a step 502, a nominal focus plane or reference plane may be determined for the specimen being examined. After the step 502, processing proceeds to a step 504 where a dither lens, according to the system described herein, is set to move at a particular resonant frequency. After the step 504, processing proceeds to a step 506 where the XY moving stage is commanded to move at a particular speed. It is noted that the order of steps 504 and 506, as with other steps of the processing discussed herein, may be appropriately modified in accordance with the system described herein. After the step 506, processing proceeds to a step 508 where sharpness calculations for focus points with respect to the specimen being examined are performed in connection with the motion (e.g., sinusoidal) of the dither lens according to the system described herein. The sharpness calculations may include use of contrast, chroma and/or other appropriate measures as further discussed elsewhere herein.

After the step 508, processing proceeds to step 510 where a best focus position is determined based on the sharpness calculations and using computed error signal information, such as the contrast error signal (CES) function, for the best focus positioning of a microscope objective used in connection with an image sensor to capture an image according to the system described herein. After the step 510, processing proceeds to a step 512 where a control signal concerning the best focus position is sent to a slow focus stage controlling the position (Z-axis) of the microscope objective. Step 512 also may include sending a trigger signal to the camera (e.g., image sensor) to capture an image of the specimen portion under the objective. The trigger signal may be a control signal causing capture of the image by the image sensor such as, for example, after a specific number of cycles (e.g. as related to the dither lens movement). After the step 512, processing proceeds to a test step 514 where it is determined whether the speed of the XY moving stage, holding the specimen under scan, should be adjusted. In an embodiment, the determination may be made according to look ahead processing techniques using sharpness and/or other information of multiple zones in a focus field of view, as further discussed in detail elsewhere herein. In other embodiments, the determination may be made based only on the use of one sharpness and/or other information for one zone without using look-ahead processing. If, at the test step 514, it is determined that the speed of the XY stage is to be adjusted, then processing proceeds to a step 516 where the speed of the XY moving stage is adjusted. After the step 516, processing proceeds back to the step 508. If, at the test step 514, it is determined that no adjustments to the speed of the XY moving stage are to be made, then processing proceeds to a test step 518 where it is determined whether focus processing is to continue. If processing is to continue, then processing back to the step 508. Otherwise, if processing is not continue (e.g., the scanning of the current specimen is complete), then focus processing is ended and processing is complete.

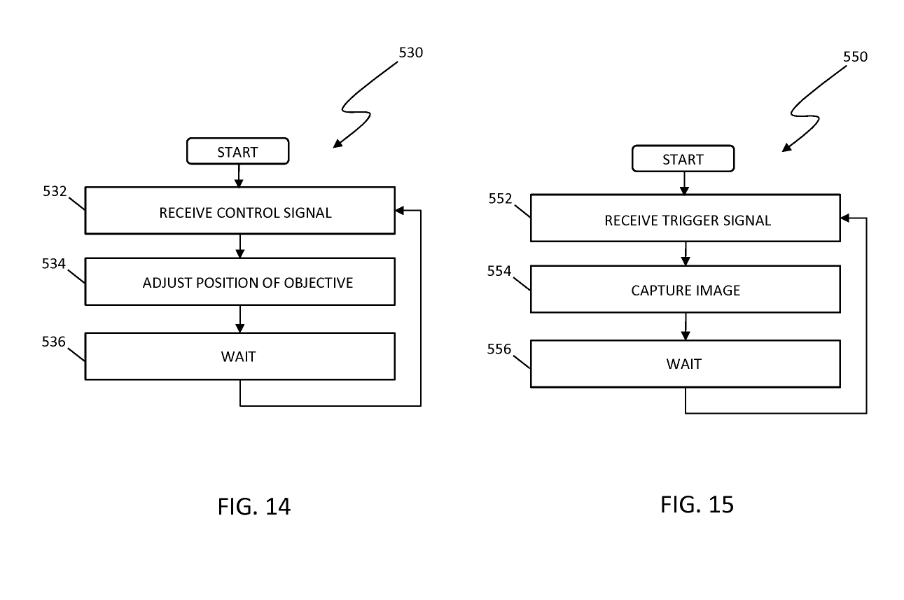

FIG. 14 is a flow diagram 530 showing processing at the slow focus stage according to an embodiment of the system described herein. At a step 532, the slow focus stage, that controls a position (e.g., along the Z-axis) of a microscope objective, receives a control signal with information for adjusting a position of the microscope objective that is examining a specimen. After the step 532, processing proceeds to a step 534 where the slow focus stage adjusts the position of the microscope objective according to the system described herein. After the step 534, processing proceeds to a waiting step 536 where the slow focus stage waits to receive another control signal. After the step 536, processing proceeds back to the step 532.

FIG. 15 is a flow diagram 550 showing image capture processing according to an embodiment of the system described herein. At a step 552, an image sensor of a camera receives a trigger signal and/or other instruction that triggers processing to capture an image of a specimen under microscopic examination. In various embodiments, the trigger signal may be received from a control system that controls triggering of the image sensor image capture processing after a specific number of cycles of motion of a dither lens used in focus processing according to the system described herein. Alternatively, the trigger signal may be provided based on a position sensor on the XY moving stage. In an embodiment, the position sensor may be a Renishaw Linear Encoder Model No. T1000-10A. After the step 552, processing proceeds to a step 554, where the image sensor captures an image. As discussed in detail herein, the captured image by the image sensor may be in focus in connection with operation of a focusing system according to the system described herein. Captured images may be stitched together in accordance with other techniques referenced herein. After the step 554, processing proceeds to a step 556 where the image sensor waits to receive another trigger signal. After the step 556, processing proceeds back to the step 552.

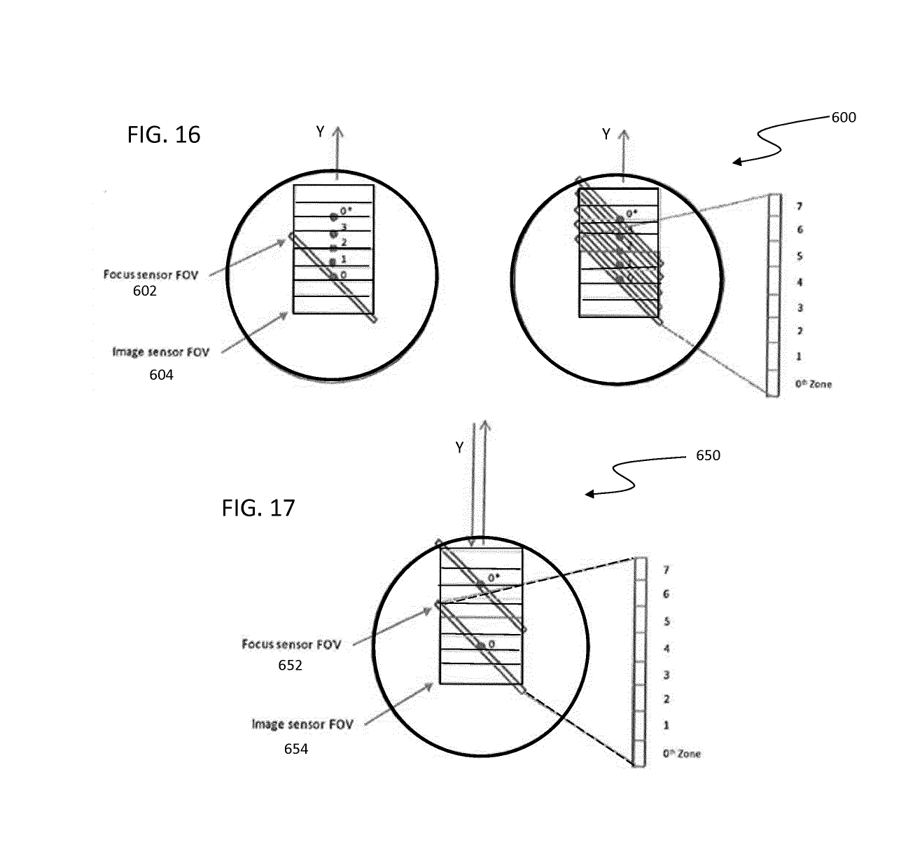

FIG. 16 is a schematic illustration 600 showing an alternative arrangement for focus processing according to an embodiment of the system described herein. A windowed focus sensor may have a frame field of view (FOV) 602 that may be tilted or otherwise positioned to diagonally scan a swath substantially equal to the width of the imaging sensor frame FOV 604. As described herein, the window may be tilted in the direction of travel. For example, the frame FOV 602 of the titled focus sensor may be rotated to 45 degrees which would have an effective width of 0.94×0.707=0.66 mm at the object (tissue). The frame FOV 604 of the imaging sensor may have an effective width of 0.588 mm, therefore, as the XY moving stage holding the tissue moves under the objective, the titled focus sensor frame FOV 602 sees the edges of the swath observed by the image sensor. In the view, multiple frames of the tilted focus sensor are shown superimposed on the image sensor frame FOV 604 at intermediate positions at times 0, 1, 2 and 3. Focus points may be taken at three points between the centers of adjacent frames in the focus column. Focus positions 0, 1, 2, and 3 are used to extrapolate the best focus position for the next snapped frame at position 0*. The scan time for this method would be similar to the methods described elsewhere herein. While the frame FOV 602 of the titled focus sensor has a shorter look ahead, in this case 0.707×(0.94−0.432)/2=0.18 mm or the tilted focus sensor encroaches 42% into the next frame to be acquired, the frame FOV 602 of the tilted focus sensor, being oblique with respect to the image sensor frame FOV 604, sees the tissue on the edges of the scan swath which may be advantageous in certain cases to provide edge focus information.

FIG. 17 is a schematic illustration 650 showing an alternative arrangement for focus processing according to another embodiment of the system described herein. As in the illustration 650, the frame FOV 652 of the titled focus sensor and the frame FOV 654 of the image sensor is shown. The frame FOV 652 of the tilted sensor may be used to acquire focus information on the forward pass across the tissue. In the backward pass the imaging sensor snaps frames while the focus stage adjusts using the prior forward pass focus data. If one wanted to take focus data at every image frame skipping intermediate positions 0, 1, 2, 3 in the prior method, the XY moving stage could move 4× the speed in the forward pass given the high rate of focus point acquisition. For example, for a 15 mm×15 mm at 20×, a column of data is 35 frames. Since the focus data is acquired at 120 points per second, the forward pass can be executed in 0.3 seconds (35 frames/120 focus points per second). The number of columns in this example is 26, therefore the focus portion can be done in 26×0.3 or 7.6 seconds. The image acquisition at 30 fps is about 32 seconds. Thus the focus portion of the total scan time is only 20%, which is efficient. Further, if focus were allowed to skip every other frame, the focus portion of the scan time would further drop substantially.

It is noted that, in other embodiments, the above-noted embodiments for positions and orientations of the focus area of the focus sensor may be used in connection with only one zone, without using a look ahead processing, in connection with the system described herein. The focus frames may, accordingly, not extend beyond the image frame and may be wider and/or otherwise larger than illustrated in the schematic illustrations 600 and 650 and instead being sized like the focus frame of illustration 300. In still other embodiments, the focus area may be positioned at other locations within the field of view, and at other orientations, to sample adjacent columns of data to provide additional focus information, including additional look ahead information, that may be used in connection with the system described herein.

The XY moving stage conveying the slide may repeat the best focus points produced on the forward travel with respect to those produced on the backward travel. For a 20×0.75 NA objective where the depth of focus is 0.9 micron, it would be desirable to repeat to about 0.1 micron. Stages may be constructed that meet 0.1 micron forward/backward repeatability and, accordingly, this requirement is technically feasible, as further discussed elsewhere herein.

In an embodiment, a tissue or smear on a glass slide being examined according to the system described herein may cover the entire slide or approximately a 25 mm×50 mm area. Resolutions are dependent on the numerical aperture (NA) of the objective, the coupling medium to the slide, the NA of the condenser and the wavelength of light. For example, at 60×, for a 0.9 NA microscope objective, plan apochromat (Plan APO), in air at green light (532 nm), the lateral resolution of the microscope is about 0.2 um with a depth of focus of 0.5 um.

In connection with operations of the system described herein, digital images may be obtained by moving a limited field of view via a line scan sensor or CCD array over the area of interest and assembling the limited field of views or frames or tiles together to form a mosaic. It is desirable that the mosaic appear seamless with no visible stitch, focus or irradiance anomalies as the viewer navigates across the entire image.

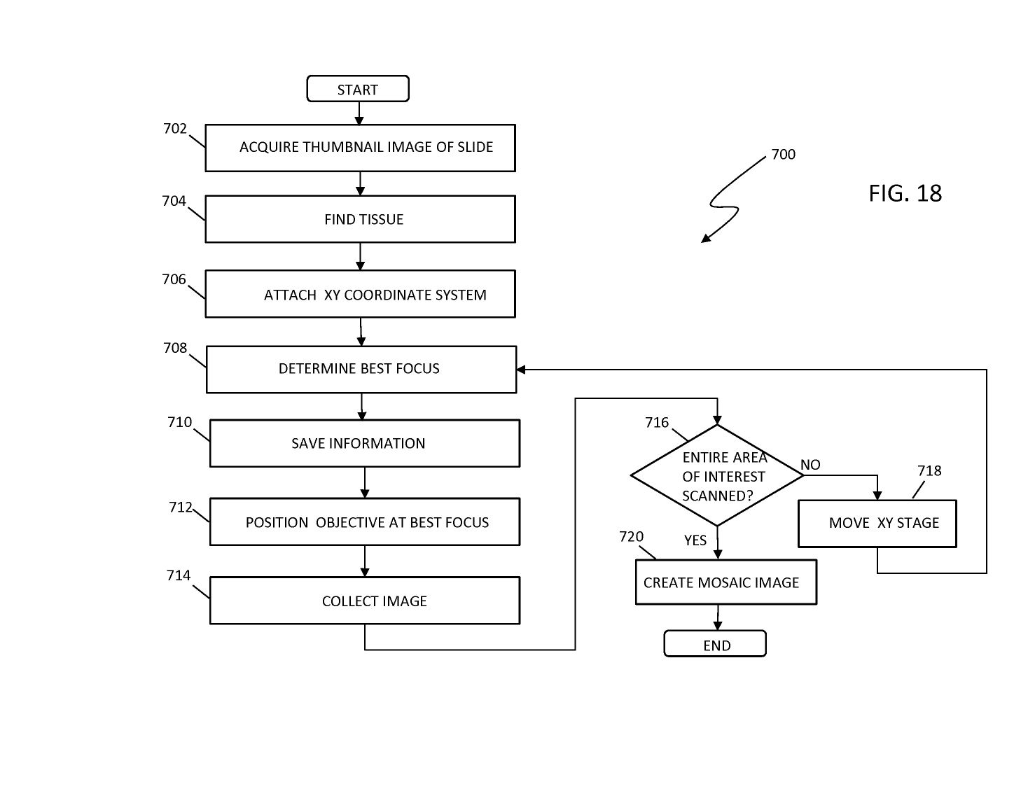

FIG. 18 is a flow diagram 700 showing processing to acquire a mosaic image of tissue on a slide according to an embodiment of the system described herein. At a step 702, a thumbnail image of the slide may be acquired. The thumbnail image may be a low resolution on the order of a 1× or 2× magnification. If a barcode is present on the slide label the barcode may be decoded and attached to the slide image at this step. After the step 702, processing proceeds to a step 704 where the tissue may be found on the slide using standard image processing tools. The tissue may be bounded to narrow the scan region to a given area of interest. After the step 704, processing proceeds to a step 706 where an XY coordinate system may be attached to a plane of the tissue. After the step 706, processing may proceed to a step 708 where one or more focus points may be generated at regular X and Y spacing for the tissue and best focus may be determined using a focus technique, such as one or more of the on-fly-focusing techniques discussed elsewhere herein. After the step 708, processing may proceed to a step 710 where the coordinates of desired focus points, and/or other appropriate information, may be saved and may be referred to as anchor points. It is noted that where frames lie between the anchor points, a focus point may be interpolated.

After the step 710, processing may proceed to a step 712 where the microscope objective is positioned at the best focus position in accordance with the techniques discussed elsewhere herein. After the step 712 processing proceeds to a step 714 where an image is collected. After the step 714, processing proceeds to a test step 716 where it is determined whether an entire area of interest has been scanned and imaged. If not, then processing proceeds to a step 718 where the XY stage moves the tissue in the X and/or Y directions according to the techniques discussed elsewhere herein. After the step 718, processing proceeds back to the step 708. If at the test step 716, it is determined that an entire area of interest has been scanned and imaged, then processing proceeds to a step 720 where the collected image frames are stitched or otherwise combined together to create the mosaic image according to the system described herein and using techniques discussed elsewhere herein (referring, for example, to U.S. Patent App. Pub. No. 2008/0240613, noted elsewhere herein). After the step 720, processing is complete. It is noted that other appropriate sequences may also be used in connection with the system described herein to acquire one or more mosaic images.

For advantageous operation of the system described herein, z positional repeatability may be repeatable to a fraction of the depth of focus of the objective. A small error in returning to the z position by the focus motor is easily seen in a tiled system (2D CCD or CMOS) and in the adjacent columns of a line scan system. For the resolutions mentioned above at 60×, a z peak repeatability on the order of 150 nanometer or less is desirable, and such repeatability would, accordingly, be suitable for other objectives, such as 4×, 20× and/or 40× objectives.

According further to the system described herein, various embodiments for a slide stage system including an XY stage are provided for pathology microscopy applications that may be used in connection with the features and techniques for digital pathology imaging that are discussed herein, including, for example, functioning as the XY moving stage 130 discussed elsewhere herein in connection with on-the-fly focusing techniques. According to an embodiment, and as further discussed in detail elsewhere herein, an XY stage may include a stiff base block. The base block may include a flat block of glass supported on raised bosses and a second block of glass having a triangular cross-section supported on raised bosses. The two blocks may be used as smooth and straight rails or ways to guide a moving stage block.

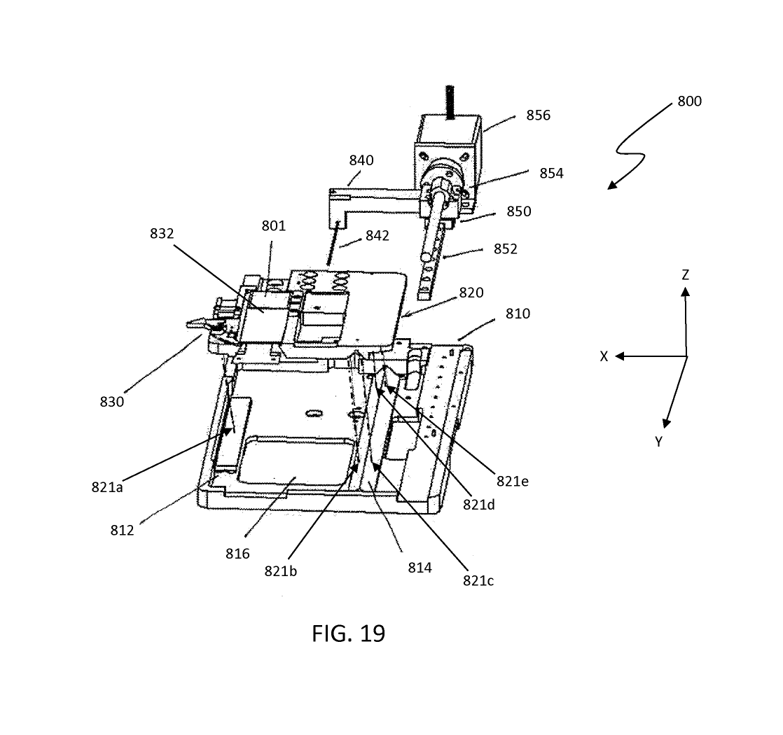

FIG. 19 is a schematic illustration showing an implementation of a precision stage 800 (e.g., a Y stage portion) of an XY stage that may be used in connection with an embodiment of the system described herein. For example, the precision stage 800 may achieve z peak repeatability on the order of 150 nanometers or less over a 25 mm×50 mm area. As further discussed elsewhere herein, the precision stage 800 may be used in connection with features and techniques discussed elsewhere herein, including, for example, functioning in connection with the XY moving stage 130 discussed with respect to the on-the-fly focusing techniques. The precision stage 800 may include a stiff base block 810 where a flat block 812 of glass is supported on raised bosses. The spacing of these bosses are such that the sag, due to the weight of the precision stage 800, of the glass blocks on the simple supports are minimized. A second block of glass 814 with a triangular cross-section is supported on raised bosses. The glass blocks 812, 814 may be adhesively bonded to the base block 810 with a semi-rigid epoxy which does not strain the glass blocks. The glass blocks 812, 814 may be straight and polished to one or two waves of light at 500 nm. A material of low thermal expansion, such as Zerodur, may be employed as a material for the glass blocks 812, 814. Other appropriate types of glass may also be used in connection with the system described herein. A cut-out 816 may allow light from a microscope condenser to illuminate the tissue on the slide.

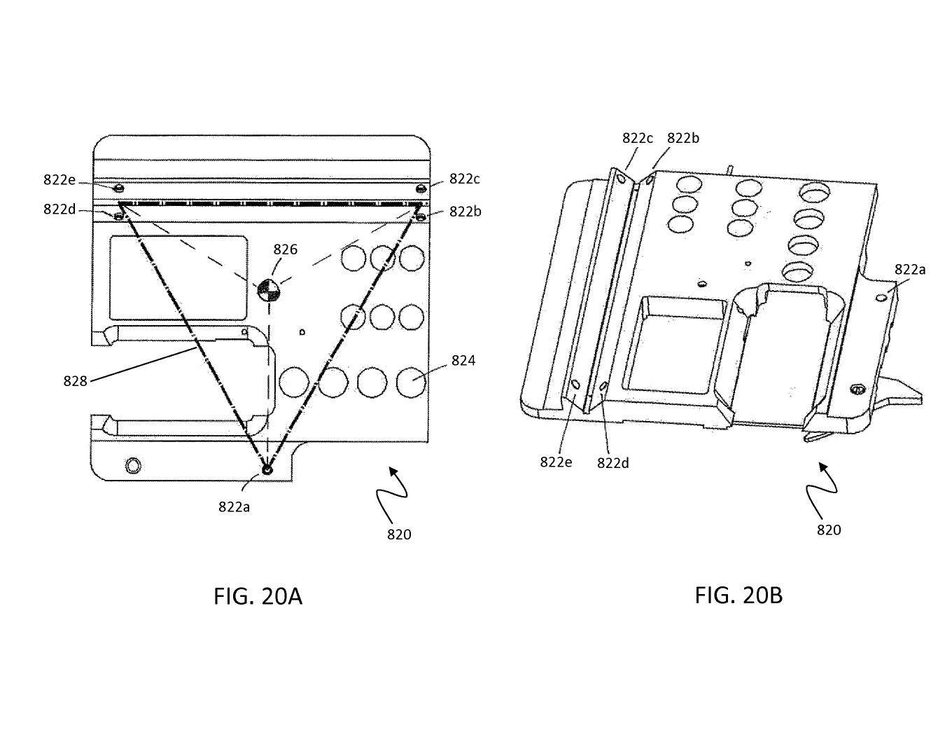

The two glass blocks 812, 814 may be used as smooth and straight rails or ways to guide a moving stage block 820. The moving stage block 820 may include hard plastic spherical shaped buttons (e.g., 5 buttons) that contact the glass blocks, as illustrated at positions 821a-e. Because these plastic buttons are spherical, the contact surface may be confined to a very small area <<0.5 mm) determined by the modulus of elasticity of the plastic. For example, PTFE or other thermoplastic blend plus other lubricant additives from GGB Bearing Technology Company, UK may be used and cast into the shape of the contact buttons of approximately 3 mm diameter. In an embodiment, the coefficient of friction between the plastic button and polished glass should be as low as possible, but it may be desirable to avoid using a liquid lubricant to save on instrument maintenance. In an embodiment, a coefficient of frictions between 0.1 and 0.15 may be readily achieved running dry. FIGS. 20A and 20B are more detailed views of the moving stage block 820 that may be used in connection with an embodiment of the system described herein and showing the spherically shaped buttons 822a-e that contact the glass blocks 810, 812 at the positions 821a-e. The buttons may be arranged in positions that allow for excellent stiffness in all directions other than the driving direction (Y). For example, two plastic buttons may face each other to contact sides of the triangular shape glass block 814 (i.e. 4 buttons 822b-e) and one plastic button 822a is positioned to contact the flat glass block 812. The moving stage block 820 may include one or more holes 824 to be light-weighted and shaped to put the center of gravity at the centroid 826 of the triangle formed by the position of plastic support buttons 822a-e. In this manner, each of the plastic buttons 822a-e at the corners of the triangle 828 may have equal weight at all times during motion of the stage 800.

In the precision stage 800, a slide 801 is clamped via a spring loaded arm 830 in the slide nest 832. The slide 801 may be manually placed in the nest 832 and/or robotically placed in the nest 832 with an auxiliary mechanism. A stiff cantilever arm 840 supports and rigidly clamps the end of small diameter flexural rod 842 that may be made of a high fatigue strength steel. In one example, this diameter may be 0.7 mm. The other end of the rod flexure 842 may be attached to the centroid location 826 on the moving stage 820. The cantilever arm 840 may be attached to a bearing block 850 which may run via a recirculating bearing design on a hardened steel rail 852. A lead screw assembly 854 may be attached to the bearing block 850 and the lead screw assembly 854 may be rotated by a stepper motor 856. Suitable components for the elements noted above may be available through several companies, such as THK in Japan. The lead screw assembly 854 drives the bearing block 850 on the rail 852 which pulls or pushes the moving stage block 820 via the rod flexure 842.

The bending stiffness of the rod flexure 842 may be a factor greater than 6000× less than the stiffness of the moving stage block 820 on its plastic pads (this is a stiffness opposing a force orthogonal to plane of the moving stage in the z direction). This effectively isolates the moving stage block 820 from up down motions of the bearing block 850/cantilever arm 840 produced by bearing noise.

The careful mass balancing and attention to geometry in design of the precision stage 800 described herein minimizes moments on the moving stage block 820 which would produce small rocking motions. Additionally, since the moving stage block 820 runs on polished glass, the moving stage block 820 has z position repeatability of less than 150 nanometer peak sufficient for scanning at 60× magnification. Since the 60× condition is the most stringent, other lower magnifications such as 20× and 40× high NA objectives also show suitable performance similar to the performance obtained under 60× conditions.

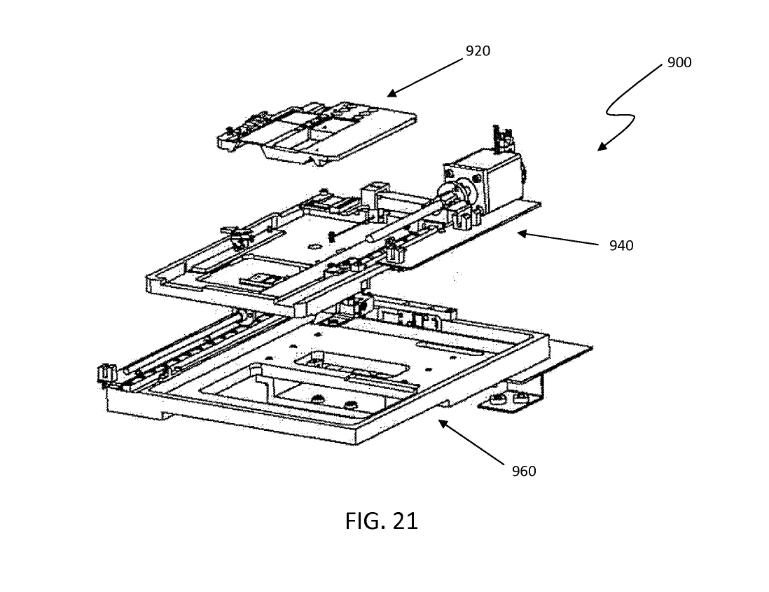

FIG. 21 shows an implementation of an entire XY compound stage 900 according to the precision stage features discussed herein and including a Y stage 920, an X stage 940 and a base plate 960 that may be used according to an embodiment of the system described herein. In this case, a base block for the Y stage 920 becomes the X stage 940 that is a moving stage in the X direction. A base block for the X stage 940 is the base plate 960 that may be fastened to ground. The XY compound stage 900 provides for repeatability in the Z direction on the order of 150 nanometer and repeatabilities on the order of 1-2 microns (or less) in the X and Y directions according to the system described herein. If the stages include feedback position via a tape-scale, such as those produced by Renishaw of Gloucestershire, England, sub-micron accuracies are achievable according to the system described herein.

The stage design according to the system described herein may be superior to spherical bearing supported moving stages in that an XY stage according to the system described herein does not suffer from repeatability errors due to non-spherical ball bearings or non-cylindrical cross roller bearings. In addition, in recirculating bearing designs, a new ball complement at different size balls may cause non-repeatable motion. An additional benefit of the embodiments described herein is the cost of the stage. The glass elements utilize standard lapping and polishing techniques and are not overly expensive. The bearing block and lead screw assembly do not need to be particularly high quality in that the rod flexure decouples the moving stage from the bearing block.

According further to the system described herein, an illumination system may used in connection with microscopy embodiments that are applicable to various techniques and features of the system described herein. It is known that microscopes may commonly use Köhler illumination for brightfield microscopy. Primary features of Köhler illumination are that the numerical aperture and area of illumination are both controllable via adjustable irises such that illumination may be tailored to a wide range of microscope objectives with varying magnification, field of view and numerical aperture. Köhler illumination offers desirable results but may require multiple components which occupy a significant volume of space. Accordingly, various embodiments of the system described herein further provide features and techniques for advantageous illumination in microscopy applications that avoid certain disadvantages of known Köhler illumination systems while maintaining the advantages of Köhler illumination.

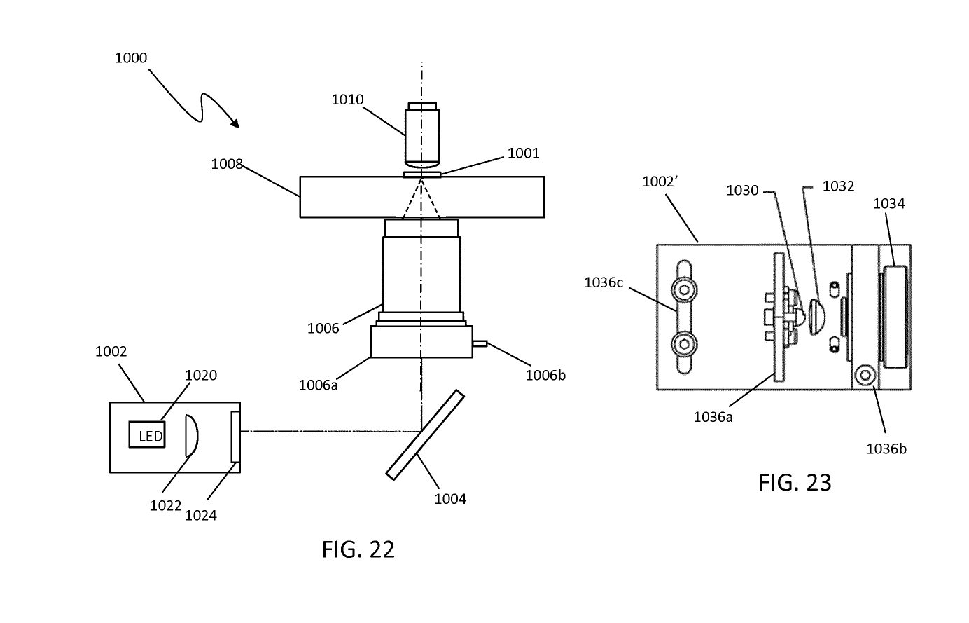

FIG. 22 is a schematic illustration showing an illumination system 1000 for illuminating a slide 1001 using a light-emitting diode (LED) illumination assembly 1002 that may be used in connection with an embodiment of the system described herein. It is noted that other appropriate illumination systems may also be used in connection with the system described herein. The LED illumination assembly 1002 may have various features according to multiple embodiments as further discussed herein. Light from the LED illumination assembly 1002 is transmitted via a mirror 1004 and/or other appropriate optical components to a condenser 1006. The condenser 1006 may be a condenser having a suitable working distance (e.g., at least 28 mm) to accommodate any required working distance of an XY stage 1008, as further discussed elsewhere herein. In an embodiment, the condenser may be condenser SG03.0701 manufactured by Motic having a 28 mm working distance. The condenser 1006 may include an adjustable iris diaphragm that controls the numerical aperture (cone angle) of light that illuminates the specimen on the slide 1002. The slide 1001 may be disposed on the XY stage 1008 under a microscope objective 1010. The LED illumination assembly 1002 may be used in connection with scanning and imaging the specimen on the slide 1001, including, for example, operations in relation to movement of an XY stage for dynamic focusing, according to the features and techniques of the system described herein.

The LED illumination assembly 1002 may include an LED 1020, such as a bright white LED, a lens 1022 that may be used as a collector element, and an adjustable iris field diaphragm 1024 that may control the area of illumination on the slide 1001. The emitting surface of the LED 1020 may be imaged by the lens 1022 onto an entrance pupil 1006a of the condenser 1006. The entrance pupil 1006a may be co-located with an NA adjusting diaphragm 1006b of the condenser 1006. The lens 1022 may be chosen to collect a large fraction of the output light of the LED 1020 and also to focus an image of the LED 1020 onto the NA adjusting diaphragm 1006b of the condenser 1006 with appropriate magnification so that the image of the LED 1002 fills the aperture of the NA adjusting diaphragm 1006b of the condenser 1006.

The condenser 1006 may be used to focus the light of the LED 1020 onto the slide 1001 with the NA adjusting diaphragm 1006b. The area of illumination on the slide 1001 may be controlled by the field diaphragm 1024 mounted in the LED illumination assembly 1002. The field diaphragm, and/or spacing between the condenser 1006 and the field diaphragm 1024, may be adjusted to image the light from the LED 1020 onto the plane of the slide 1001 so that the field diaphragm 1024 may control the area of the slide 1001 that is illuminated.

Since an image sensor acquires frames while a Y stage containing a slide is moving, the LED 1020 may be pulsed on and off (e.g., strobed) to allow very high brightness over a short time. For example, for a Y stage moving at about 13 mm/sec, to maintain no more than 0.5 pixel (0.250 micron/pixel) blur, the LED 1020 may be pulsed to be on for 10 microseconds. The LED light pulse may be triggered by a master clock locked to the dither lens resonant frequency in accordance with the focus system and techniques further discussed elsewhere herein.

FIG. 23 is a schematic illustration showing a more detailed side view of an embodiment for a LED illumination assembly 1002′ that may be used in connection with an embodiment of the system described herein and corresponding to the features described herein with respect to the LED illumination assembly 1002. An implementation and configuration of an LED 1030, a lens 1032, and a field diaphragm 1034 are shown with respect to and in connection with other structural support and adjustment components 1036.

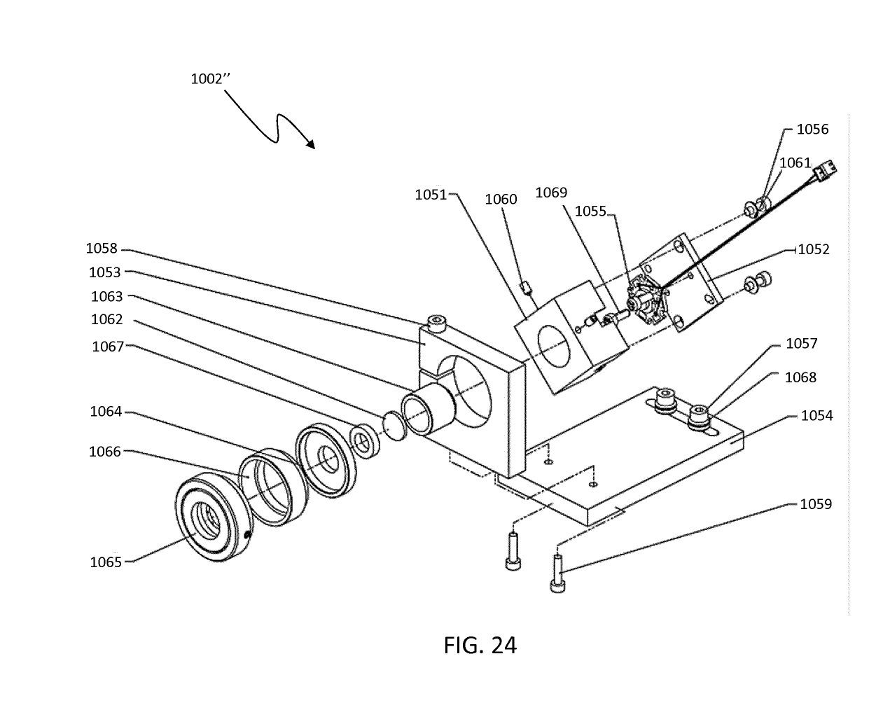

FIG. 24 is a schematic illustration showing an exploded view of a specific implementation of an LED illumination assembly 1002″ that may be used in connection with an embodiment of the system described herein having features and functions like that discussed with respect to the LED illumination assembly 1002. An adapter 1051, mount 1052, clamp 1053, and mount 1054 may be used to securely mount and situate an LED 1055 in the LED illumination assembly 1002″ so as to be securely positioned with respect to a lens 1062. Appropriate screw and washer components 1056-1061 may be further used to secure and mount the LED illumination assembly 1002″. In various embodiments, the LED 1055 may be a Luminus, PhatLight White LED CM-360 Series this is a bright white LED having an optical output of 4,500 lumens and long life of 70,000 hours and/or a suitable LED made by Luxeon. The lens 1062 may be an MG 9P6 mm, 12 mm OD (outer diameter) lens. A tube lens component 1063, adapter 1064, stack tube lens component and retaining ring 1067 may be used to position and mount the lens 1062 with respect to the adjustable field diaphragm component 1065. The adjustable field diaphragm component 1065 may be a Ring-Activated Iris Diaphragm, part number SM1D12D by Thor Labs. The stack tube lens 1066 may be a P3LG stack tube lens by Thor Labs. The tube lens 1063 may be a P50D or P5LG tube lens by Thor Labs. Other washer 1068 and screw components 1069 may be used, where appropriate, to further secure and mount elements of the LED illumination assembly 1002″.

Various embodiments discussed herein may be combined with each other in appropriate combinations in connection with the system described herein. Additionally, in some instances, the order of steps in the flowcharts, flow diagrams and/or described flow processing may be modified, where appropriate. Further, various aspects of the system described herein may be implemented using software, hardware, a combination of software and hardware and/or other computer-implemented modules or devices having the described features and performing the described functions. Software implementations of the system described herein may include executable code that is stored in a non-transitory computer readable medium and executed by one or more processors. The non-transitory computer readable medium may include a computer hard drive, ROM, RAM, flash memory, portable computer storage media such as a CD-ROM, a DVD-ROM, a flash drive and/or other drive with, for example, a universal serial bus (USB) interface, and/or any other appropriate tangible storage medium or computer memory on which executable code may be stored and executed by a processor. The system described herein may be used in connection with any appropriate operating system.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

1. A device for obtaining a focused image of a specimen, comprising:
an objective lens disposed for examination of the specimen;
a slow focusing stage coupled to the objective lens, wherein the slow focusing stage controls movement of the objective lens;
a dither focus stage including a dither lens, wherein the dither focus stage moves the dither lens;
a focus sensor that provides focus information in accordance with light transmitted via the dither lens;
at least one electrical component that uses the focus information to determine a metric and a first focus position of the objective lens in accordance with the metric, wherein the at least one electrical component includes an error signal component that processes error signal information generated based on the metric to determine the first focus position, wherein the at least one electrical component sends position information to the slow focusing stage for moving the objective lens into the first focus position; and
an image sensor that captures an image of the specimen after the objective lens is moved into the first focus position.

2. The device according to claim 1, wherein the error signal information is determined according to an error signal function using points of a waveform generated based on the metric according to the motion of the dither lens.

3. The device according to claim 2, wherein the error signal function is a contrast error signal function, and wherein the first focus position is determined where the contrast error signal function is zero.

4. The device according to claim 3, wherein the contrast error signal function is determined based on at least three points of a sharpness waveform computed for each of at least one position on a sharpness response curve where the motion of the dither lens is centered.

5. The device according to claim 4, wherein the contrast error signal (CES) may be represented by an equation:
CES=(a−c)/b,
where a is a trough of the sharpness waveform, b is a peak of the sharpness waveform, and c is a subsequent trough of the sharpness waveform.

6. The device according to claim 1, further comprising:
an XY moving stage, wherein the specimen is disposed on the XY moving stage, and wherein at least one of the following is provided: (i) the at least one electrical component controls movement of the XY moving stage, or (ii) the XY moving stage is phase locked with the motion of the dither lens.

7. The device according to claim 1, wherein the dither focus stage includes a voice-coil actuated flexured assembly that moves the dither lens in a translational motion.

8. The device according to claim 1, wherein the dither lens is moved at a resonant frequency that is at least 60 Hz, and wherein the at least one electrical component uses the focus information to perform at least 60 focus calculations per second.

9. The device according to claim 1, wherein the focus sensor and the dither focus stage are set to operate bidirectionally, wherein the focus sensor produces the focus information on both an up and down portion of a sinusoid waveform of the motion of the dither lens at the resonant frequency.

10. The device according to claim 1, wherein the metric includes at least one of: contrast information, sharpness information, and chroma information.

11. A method for obtaining a focused image of a specimen, comprising:
controlling movement of an objective lens disposed for examination of the specimen;
controlling motion of a dither lens;
providing focus information in accordance with light transmitted via the dither lens;
using the focus information to determine a metric and determine a first focus position of the objective lens in accordance with the metric, wherein determining the first focus position includes processing error signal information generated based on the metric;
sending position information that is used to move the objective lens into the first focus position.

12. The method according to claim 11, wherein the error signal information is determined according to an error signal function using points of a waveform generated based on the metric according to the motion of the dither lens.

13. The method according to claim 12, wherein the error signal function is a contrast error signal function, and wherein the first focus position is determined where the contrast error signal function is zero.

14. The method according to claim 13, wherein the contrast error signal function is determined based on at least three points of a sharpness waveform computed for each of at least one position on a sharpness response curve where the motion of the dither lens is centered.

15. The method according to claim 14, wherein the contrast error signal (CES) may be represented by an equation:
CES=(a−c)/b,
where a is a trough of the sharpness waveform, b is a peak of the sharpness waveform, and c is a subsequent trough of the sharpness waveform.

16. The method according to claim 11, wherein the first focus position is determined as a best focus position, and the method further comprising:
capturing an image of the specimen after the objective lens is moved into the best focus position.

17. The method according to claim 11, wherein the dither lens is moved at a resonant frequency that is at least 60 Hz, and wherein at least 60 focus calculations are performed per second.

18. The method according to claim 11, wherein the metric includes at least one of: sharpness information, contrast information and chroma information.

19. A non-transitory computer readable medium storing software for obtaining a focused image of a specimen, the software comprising:
executable code that controls movement of an objective lens disposed for examination of the specimen;
executable code that controls motion of a dither lens;
executable code that provides focus information in accordance with light transmitted via the dither lens;
executable code that uses the focus information to determine a metric and determine a first focus position of the objective lens in accordance with the metric, wherein determining the first focus position includes processing error signal information generated based on the metric; and
executable code that sends position information that is used to move the objective lens into the first focus position.

20. The non-transitory computer readable medium according to claim 19, wherein the error signal information is determined according to an error signal function using points of a waveform generated based on the metric according to the movement of the dither lens, wherein the error signal information is determined according to an error signal function using points of a waveform generated based on the metric according to the movement of the dither lens, wherein the error signal function is a contrast error signal function, and wherein the first focus position is determined where the contrast error signal function is zero, wherein the contrast error signal function is determined based on at least three points of a sharpness waveform computed for each of at least one position on a sharpness response curve where the movement of the dither lens is centered.

21. A device for obtaining a focused image of a specimen, comprising:
an objective lens disposed for examination of the specimen;
a slow focusing stage coupled to the objective lens, wherein the slow focusing stage controls movement of the objective lens;
a dither focus stage including a dither lens, wherein the dither focus stage moves the dither lens;
a focus sensor that provides focus information in accordance with light transmitted via the dither lens;
at least one electrical component that uses the focus information to determine a metric and a first focus position of the objective lens in accordance with the metric, wherein the at least one electrical component includes an error signal component that processes error signal information generated based on the metric to determine the first focus position, wherein the at least one electrical component sends position information to the slow focusing stage for moving the objective lens into the first focus position; and
an image sensor that captures an image of the specimen on a column-by-column basis, during a scanning of the specimen in a serpentine manner, and wherein when a first column of the specimen is scanned in a first direction, a field of view of the focus sensor is aligned with a second column that is adjacent to the first column, such that focus data of the second column is generated.

22. The device of claim 21, further comprising scanning the second column, in a direction that is reverse to the first direction that the first column was scanned, using the focus data of the second column.

23. The device of claim 22, wherein focus data of the first column is predetermined, and wherein the objective lens is moved into a second focus position when the focus data of the first column differs from the focus data of the second column.
